(19) United States
(12) Patent Application Publication
(10) Pub. No.: US 2004/0126393 A1
(43) Pub. Date: Jul. 1, 2004

(54) Infection preventive or therapeutic agent and food

(76) Inventors: Tatsuo Suzuki, Tokyo (JP); Kenichi Matsunaga, Tokorozawa-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/695,766

(22) Filed: Oct. 30, 2003

(30) Foreign Application Priority Data
Dec. 27, 2002 (JP) 2002-381274

(51) Int. Cl. A61K 35/84

(52) U.S. Cl. 424/195.15

(57) ABSTRACT

An infection preventive or therapeutic agent and food are disclosed, which contain as an active ingredient an anion exchange resin adsorption fraction of a mixture obtained by mixing a hot water extract of mycelia of basidiomycetes belonging to the genus Tricholoma, in particular the Tricholoma matsutake FERM BP-7304 strain, with an alkaline solution extract of a mycelia residue when the mycelia hot water extract is obtained. The anion exchange resin adsorption fraction has a carbohydrate content of 60% to 72% in glucose equivalent determined by a phenol-sulfuric acid method and (b) a protein content of 28% to 40% in albumin equivalent determined by a copper-Folin method. Methods of preventing or treating infection(s) with a pathogenic microorganism by the use of the preventive or therapeutic agent and food are also disclosed.

### BACKGROUND OF THE INVENTION

1. Field of the Invention

The present invention relates to a preventive or therapeutic agent and food used to combat pathogenic microorganisms, in particular pathogenic bacteria, harmful to animals and humans. The infection preventive or therapeutic agent and food of the present invention may be administered not only as a medicament but also in various forms, for example, as eatable and drinkable products such as health-promoting foods (specified health food and nutritional-functional food), as so-called health food (both including drinkable products), or as feeds. Further, the agent of the present invention may be administered in the form of an agent that is temporarily kept in the mouth but then spat out without the retention of most components, for example, a dentifrice, a mouthwash agent, a chewing gum, or a collutorium, or in the form of an inhalant drawn in through the nose.

2. Description of Related Art

It is known that matsutake [Tricholoma matsutake (S. Ito & Imai) Sing.], which is a member of basidiomycetes belonging to the genus Tricholoma, contains various physiologically active substances. For example, JP-B-57-1230(Kokoku) discloses emitanine-5-A, emitanine-5-B, emitanine-5-C, and emitanine-5-D, which are separated and purified from a liquid extract obtained by extracting a liquid culture of Tricholoma matsutake mycelia with hot water or a diluted alkaline solution, exhibit activity of inhibiting the proliferation of sarcoma 180 cells. Further, JP Patent No. 2767521 discloses that a protein with a molecular weight of 0.2 to 0.21 million (a molecular weight of a subunit=0.1 to 0.11 million) that is separated and purified from an extract of Tricholoma matsutake fruit bodies with water exhibits an titumor activity.

Furthermore, the present inventors have found that a hot water extract of Tricholoma matsutake, an alkali-solution extract of Tricholoma matsutake, or an adsorption fraction of these extracts by an anion exchange resin has immuno-enhancing activity (WO 01/49308 pamphlet). The present inventors have also found that a partial purified fraction derived from particular mycelia of Tricholoma matsutake has activity of promoting recovery from stress loading (PCT WO 03/070264 A1).

### SUMMARY OF THE INVENTION

As described above, it has been found that Tricholoma matsutake has various physiological activities such as antitumor activity, immuno-enhancing activity, and activity of promoting recovery from stress loading. However, as far as the present inventors know, it has not yet been reported that Tricholoma matsutake, or basidiomycetes belonging to Tricholoma, which is a genus of Tricholoma matsutake, has excellent preventive or therapeutic effects for the onset and/or the progression of the infection with pathogenic bacteria such as Pseudomonas aeruginosa and Listeria monocytogenes.

The present inventors have newly discovered that Tricholoma matsutake or basidiomycetes belonging to Tricholoma, which is a genus of the matsutake, has excellent preventive or therapeutic effects for fighting infection with pathogenic bacteria such as Pseudomonas aeruginosa and Listeria monocytogenes. They have thereby completed the present invention.

Hence, an object of the present invention is to provide a preventive or therapeutic agent utilizing basidiomycetes belonging to the genus Tricholoma such as Tricholoma matsutake.

Another object of the present invention is to provide a preventive or therapeutic food utilizing basidiomycetes belonging to the genus Tricholoma such as Tricholoma matsutake.

Still another object of the invention is to provide a method of preventing or treating infection(s) with a pathogenic microorganism by the administration of the preventive or therapeutic agent.

A further object of the invention is to provide a method of preventing or treating infection(s) with a pathogenic microorganism by the intake of the preventive or therapeutic food.

The present invention relates to a preventive or therapeutic agent for infections with pathogenic microorganisms containing basidiomycetes belonging to the genus Tricholoma or extracts thereof.

Further, the present invention relates to a preventive or therapeutic food for infections with pathogenic microorganisms containing basidiomycetes belonging to the genus Tricholoma or extracts thereof.

Further, the present invention relates to a method of preventing or treating infection(s) with a pathogenic microorganism which comprises administrating to a human or an animal in an effective amount of the preventive or therapeutic agent.

Still further, the present invention relates to a method of preventing or treating infection(s) with a pathogenic microorganism which comprises the intake of by a human or an animal in an effective amount of the preventive or therapeutic food.

### BRIEF DESCRIPTION OF THE DRAWINGS

FIG. 1 illustrates a spectrum obtained by a ¹H one-dimensional NMR measurement of an adsorption fraction M2, which is an embodiment used for the invention.

FIG. 2 illustrates a spectrum obtained by a ¹³C one-dimensional NMR measurement of the adsorption fraction M2, which is an embodiment used for the invention.

FIG. 3 illustrates a spectrum (broad) obtained by a ¹³C one-dimensional NMR measurement of the adsorption fraction M2, which is an embodiment used for the invention.

FIG. 4 illustrates a CD spectrum obtained by a circular dichroism analysis of the adsorption fraction M2, which is an embodiment used for the invention.

FIG. 5 illustrates a spectrum obtained by an infrared spectroscopic analysis of the adsorption fraction M2, which is an embodiment used for the invention.

FIG. 6 illustrates a spectrum obtained by an ultraviolet spectroscopic analysis of the adsorption fraction M2, which is an embodiment used for the invention.

FIG. 7 illustrates a spectrum obtained by an ESR analysis of the adsorption fraction M2, which is an embodiment used for the invention.

FIG. 8 illustrates a spectrum (broad) obtained by an ESR analysis of the adsorption fraction M2, which is an embodiment used for the invention.

### DETAILED DESCRIPTION OF THE INVENTION

Genus Tricholoma to be used for an infection preventive or therapeutic agent and food of the present invention contains basidiomycetes belonging to Tricholamataceae. Examples thereof include Tricholoma matsutake [(S. Ito & Imai) Sing.], T. fulvocastaneum Hongo sp. nov., T. bakamatsutake Hongo sp. nov., and T. muscarinum Kawamura. Among all, T. matsutake [(S. Ito & Imai) Sing.] is preferably used for the present invention.

T. matsutake can be used in any form of mycelia, broths, or fruit bodies and they can be used in either a fresh or dried state. In the present invention, fruit bodies include spores. Further, extracts from these mycelia, broths, and fruit bodies, or anion-exchange resin adsorption fraction thereof, may be used for the present invention.

In the present invention, the T. matsutake FERM BP-7304 strain is particularly preferably used, but any T. matsutake strains other than this strain may be arbitrarily used. Examples of the other strains include CM 627-3, CM 627-5, CM 627-6, CM 627-7 (available from Kureha Chemical Industry Co., Ltd.), MAFF 460031, MAFF 460033, MAFF 460034, MAFF 460035, MAFF 460036, MAFF 460037, MAFF 460038, MAFF 460040, MAFF 460041, MAFF 460042, MAFF 460096 (available from National Institute of Agrobiological Sciences, Ministry of Agriculture, Forestry, and Fishery), IFO 6929, IFO 6931, IFO 6932, IFO 6934, IFO 6915, IFO 6916, IFO 6917, IFO 6918, IFO 6919, IFO 6921, IFO 6922, IFO 6923, IFO 6924, IFO 6925, IFO 6926, IFO 6928, IFO 6930, IFO 6935, IFO 30605, IFO 30606 (available from Institute for Fermentation, Osaka), ATCC 34979, ATCC 34981, and ATCC 34988 (available from American Type Culture Collection). However, the strains to be used are not limited these examples.

The T. matsutake FERM BP-7304 strain was previously filed by the present applicant as a novel strain (PCT WO 02/30440 A1), and was deposited on Sep. 14, 2000, at Independent Administrative Institution, National Institute of Advanced Industrial Science and Technology (former National Institute of Bioscience and Human-Technology, Agency of Industrial Science and Technology, Japan). This T. matsutake FERM BP-7304 strain was a mycelium passage strain obtained by cutting out a fruit body tissue from the T. matsutake CM 6271 strain harvested in Kameoka, Kyoto, Japan, and culturing the tissue in a test tube. The FERM BP-7304 strain has been maintained in Biomedical Research Laboratories, Kureha Chemical Industries Co., Ltd.

The fruit body of the T. matsutake FERM BP-7304 strain had a fruit body form identical to a T. matsutake fruit body described on plate pages 9 and 26 of “Genshoku-nihon shin-kinrui zukan (1)” (edited by Rokuya Imaseki and Tsuguo Hongo, published by Hoikusha in 1957).

The T. matsutake FERM BP-7304 strain can be subcultured in a slant Ebios agar medium. After mycelia of the T. matsutake FERM BP-7304 strain is inoculated in a plate Ebios agar medium, white mycelia densely grow in a radial pattern, forming a large colony. When the colony is observed with a scanning electron microscope, an uncountable number of branched mycelia with a thickness of 1 to 2 μm are present and sometimes projections with a size of several μm are present on the side of the mycelia. For mass cultivation of the mycelia of the strain, the mycelia are inoculated on a liquid medium and cultured by stationary cultivation, shaking cultivation, tank cultivation, or the like.

It should be noted that the T. matsutake FERM BP-7304 strain can be maintained by subculture or cultured mostly in the form of mycelia, but it may also exist in the form of fruit body.

The mycological characteristics of the T. matsutake FERM BP-7304 strain are described below.

(1) Cultural and morphological characteristics in malt extract agar medium:

White hyphae grew densely and radially, forming a colony. The diameter of the colony on the 30th day after inoculation was about 4 cm.

(2) Cultural and morphological characteristics in Czapeck agar medium, oatmeal agar medium, synthetic mucor agar-medium, and phenoloxidase reaction assay medium:

Almost no growth of hyphae was observed in any of the above media even after 1 month had passed since inoculation.

(3) Cultural and morphological characteristics in YpSs agar medium:

The T. matsutake FERM BP-7304 strain grew in a mat shape having a white gloss. On the 30th day after inoculation, the growth distance was about 5 mm.

(4) Cultural and morphological characteristics in glucose dry yeast agar medium:

The T. matsutake FERM BP-7304 strain grew in a mat shape having a white gloss. On the 30th day after inoculation, the growth distance was about 2 mm.

(5) Optimum growth temperature and growth range:

In a 100-mL Erlenmeyer flask containing 10 mL of sterilized liquid medium (3% glucose, 0.3% yeast extract, pH 7.0), about 2 mg of seed fungi of the T. matsutake FERM BP-7304 strain was each inoculated and cultured at various temperatures of 5 to 35° C. On 28th day of incubation, fungus bodies were taken out from the flask, washed well with distilled water, and then dried for mass measurement. The results show that the mass of the fungus bodies linearly increased within the temperature range of 5 to 15° C. and leniently increased within the temperature range of 15 to 25° C. Almost no fungi grew at temperatures of 27.5° C. or more. The optimum temperature for growth is from 15 to 25° C.

(6) Optimum growth pH and growth range:

Liquid media (3% glucose, 0.3% yeast extract) were adjusted with 1 mol/L hydrochloric acid or 1 mol/L potassium hydroxide so that the media having various pH levels from 3.0 to 8.0 were prepared to determine the pH for fungus body growth. Namely, each medium was sterilized with a filter, and 10 mL of the sterilized medium was dispensed into a 100-mL sterilized Erlenmeyer flask. About 2 mg of seed fungi of the T. matsutake FERM BP-7304 strain was inoculated in the flask and cultured at 22° C. Thereafter, fungus bodies were taken out from the flask, washed well with distilled water, and then dried for mass measurement. The results show that the pH growth limit for the fungus bodies was from 3.0 to 7.0 and the optimum pH for growth was 4.0 to 6.0.

(7) Formation of zone line by dual culture:

On an Ebios plate agar medium, a block (about 3 mm×3 mm×3 mm) of the T. matsutake FERM BP-7304 strain and each block (about 3 mm×3 mm×3 mm) of 13 kinds of known T. matsutake strains (for example, IFO 6915 strain; Institute for Fermentation Osaka) were placed with about 2 cm of distance between each strain, and cultured at 22° C. for 3 weeks. Thereafter, it was determined whether a zone line was formed on the boundary between two colonies among them.

The results show that the T. matsutake FERM BP-7304 strain did not form definite zone lines against all of the known T. matsutake strains (13 kinds). It is considered that no zone line is formed by dual culture between different strains of T. matsutake, and among the known T. matsutake strains (13 kinds) there was no combination of strains that formed a definite zone line therebetween. Therefore, it is considered the strains are compatible one another.

(8) Nutritional requirement:

About 2 mg of seed fungi of the T. matsutake FERM BP-7304 strain was inoculated in a 100-mL Erlenmeyer flask containing 10 mL of sterilized synthetic medium for mycorrhizal fungus (Ohta medium, Ohta et al. “Trans. Mycol. Soc. Jpn., 31, 323-334, 1990), and cultured at 22° C. On 42nd day of culturing, fungus bodies were taken out from the flask, washed well with distilled water, and dried for mass measurement. Consequently, 441 mg of fungus body was obtained.

Instead of glucose in the above synthetic medium for mycorrhizal fungus as a carbon (C) source, any one of 28 kinds of carbohydrate-related substances was added to each medium. The T. matsutake FERM BP-7304 strain was inoculated and cultured on each medium, and after the completion of culture the mass of fungus bodies was measured. As a result, the carbohydrate-related substances are listed below in descending order corresponding to the fungus body mass:

Wheat starch>corn starch>dextrin>methyl β glucoside>cellobiose>mannose>fructose>arabinose>sorbitol>glucose>lactose>glycogen>mannitol>ribose>maltose>trehalose>galactose>raffinose>melibiose>N-acetylglucosamine.

Incidentally, almost no growth of the fungi was observed in cellulose, dulcitol, sucrose, xylose, methyl a glucoside, inulin, inositol, or sorbose.

Next, instead of ammonium tartrate in the above synthetic medium for mycorrhizal fungus as a nitrogen (N) source, any one of 17 kinds of nitrogen-related substances was added to each medium. The T. matsutake FERM BP-7304 strain was inoculated and cultured on each medium, and after the completion of culture the mass of fungus bodies was measured. As a result, the nitrogen-related substances are listed below in descending order corresponding to the fungus body mass:

Corn steep liquor>soy peptone>milk peptone>ammonium nitrate>ammonium sulfate>ammonium tartrate>ammonium carbonate>asparagine>ammonium phosphate>ammonium chloride>sodium nitrate>meat extract>yeast extract>casamino acid>chlorella>triptone>potassium nitrate.

Further, among minerals and vitamins in the above synthetic medium, a medium was prepared without a particular single component. The T. matsutake FERM BP-7304 strain was inoculated and cultured on that medium, and after the completion of culture the mass of fungus bodies was measured.

As a result, even when any one of calcium chloride-dihydrate, manganese (II) sulfate-pentahydrate, zinc sulfateheptahydrate, cobalt sulfate-heptahydrate, copper sulfate-pentahydrate, nickel sulfate-hexahydrate, amine hydrochloride, nicotinic acid, folic acid, biotin, pyridoxine hydrochloride, carnitine chloride, adenine sulfatedihydrate, and choline hydrochloride was removed from the medium, the fungus body mass was almost uneffected.

On the other hand, when any one of magnesium sulfateheptahydrate, iron (II) chloride, and potassium dihydrogen phosphate was removed, the fungus body mass remarkably reduced. In other words, magnesium, iron, phosphorus, and potassium are considered essential for the growth of the T. matsutake FERM BP-7304 strain.

(9) DNA base composition (GC content):

The GC content was 49.9%.

(10) DNA pattern prepared by RAPD method:

In terms of DNA patterns prepared by the RAPD (Random Amplified Polymorphic DNA) method independently using 6 different kinds of PCR (Polymerase Chain Reaction) primers (10 mer), the T. matsutake FERM BP-7304 strain was compared with 44 kinds of known T. matsutake strains (for example, the IFO 6915 strain; Institute for Fermentation Osaka). The T. matsutake FERM BP-7304 strain exhibited a DNA pattern different from all of the other known T. matsutake strains (44 kinds).

Preferable embodiments of the infection preventive or therapeutic agent and food according to the present invention contain as an active ingredent: (i) T. matsutake FERM BP-7304 strain (e.g., mycelia, broths, or fruit bodies of the strain); (ii) a hot water extract of T. matsutake FERMBP-7304 strain (e.g., hot water extract of mycelia, broths, or fruit bodies of the strain); (iii) an alkaline solution extract of T. matsutake FERM BP-7304 strain (e.g., alkaline solution extract from mycelia, broths, or fruit bodies of the strain); (iv) an anion resin adsorption fraction of a hot water extract or an alkaline solution extract of T. matsutake FERM BP-7304 strain (e.g., anion resin adsorption fraction of a hot water extract or an alkaline solution extract of mycelia, broths, or fruit bodies of the strain); or (v) an anion exchange resin adsorption fraction of a liquid mixture obtained by mixing a hot water extract of mycelia of T. matsutake FERM BP-7304 strain with an alkaline solution extract of mycelia residue when the mycelia hot water extract is obtained, wherein (a) the carbohydrate content in glucose equivalent by a phenol-sulfuric acid method is 60% to 72% and (b) the protein content in albumin equivalent by a copper-Folin method is 28% to 40%. However, the active ingredient is not limited to these embodiments.

For the present invention, the above embodiment (v) is preferable. It should be noted that an anion exchange resin adsorption fraction (M2 fraction) described as the embodiment (v), and an immuno-enhancing agent and an agent for promoting recovery from stress loading containing the fraction have been described in the application (PCT WO 03/070264 A1) previously filed by the present applicant.

The anion exchange resin adsorption fraction described as above (v) can be prepared, for example, by a production method comprising:

a step for culturing the T. matsutake FERM BP-7304 strain by tank culture and obtaining mycelia (hereinafter referred to as “cultivation step”)

a step for extracting the obtained mycelia of the T. matsutake FERM BP-7304 strain with hot water and obtaining a mycelia hot water extract (hereinafter referred to as “hot water extraction step”);

a step for extracting a residue of the mycelia after the hot water extraction with an alkaline solution and obtaining an alkaline solution extract of the residue of the mycelia (hereinafter referredtoas “alkalinesolution extraction step”);

a step for adsorbing with an anion exchange resin an extract mixture obtained by mixing the mycelia hot water extract and the alkaline solution extract of the residue of the mycelia (hereinafter referred to as “anion exchange resin adsorption step”); and

a step for eluting an adsorption fraction with an appropriate eluting solution (hereinafter referred to as “elution step”).

However, the method is not limited thereto.

Each step of the above method can be conducted, for example, as follows, but the steps are not limited thereto.

[Cultivation Step]

The cultivation step is not particularly limited, and any of the ordinary methods for culturing T. matsutake fungi can be used. However, a method, for example, disclosed in JP Patent Application No. 2002-311840 is preferably employed, since the method enables mass production without the loss of the physiological activities of matsutake fungi. The method comprises: a step for obtaining matsutake fungi II by culturing or preserving the T. matsutake FERM BP-7304 strain (“matsutake fungi I”) in a solid or liquid medium; a step for obtaining matsutake fungi III by stationary liquid-cultivation of the matsutake fungi II; a step for obtaining matsutake fungi IV by shaking cultivation of the matsutake fungi III; a step for obtaining matsutake fungi V by stirring-culture of the matsutake fungi IV with the use of a small culture apparatus with a volume of less than 100 L without the aeration in a liquid medium; a step for obtaining matsutake fungi VI by deep stirring-culture of the matsutake fungi V with the use of a medium- or large-sized culture apparatus with a volume of 100 L or more; a step for obtaining matsutake fungi VII by deep stirring-culture of the matsutake VI with the use of a medium- or large-sized culture apparatus with a volume of 100 L or more; and a step for obtaining matsutake fungi VIII by deep stirring-culture of the matsutake fungi VII with the use of a medium- or large-sized culture apparatus with a volume of 100 L or more.

<Step for Obtaining matsutake Fungi II by Culturing or Preserving matsutake Fungi I>

A medium to be used herein is not particularly limited, as long as such medium is a common one containing a nutrient substrate for culturing matsutake fungi. Examples thereof include an Ohta medium (Ohta et al., “Trans. Mycol. Soc. Jpn.,” 31, 323-334, 1990), an MMN medium (Marx, D. H., “Phytopathology,” 59: 153-163, 1969), and a Hamada medium (Hamada, “Matsutake,” 97-100, 1964), but the usable medium is not limited to these examples.

Preferable examples of a solidifying agent for a solid medium include carrageenan, mannnan, pectin, agar, curdlan, starch, and alginic acid. Among these, agar is preferable.

Examples of usable nutrient substrate for a medium include a carbon source, a nitrogen source, and an inorganic element source.

Examples of the above carbon source include: starches, such as rice starch, wheat flour starch, potato starch, and sweet potato starch; polysaccharides, such as dextrin and amylopectin; oligosaccharides, such as maltose and sucrose; and monosaccharides, such as fructose and glucose. Examples thereof further include malt extracts. Depending on the growth speed of matsutake fungi, matsutake has a period in which monosaccharides such as glucose are preferably used and a period in which starches are preferably used. Therefore, a suitable carbon source is selected based on the period, and if necessary, these carbon sources may be used in combination.

Examples of the above nitrogen source include naturally occurring substances such as yeast extracts, dried yeast, corn steep liquor, soy flour, and soy peptone, ammonium nitrate, ammonium sulfate, and urea. These may be used either alone or in combination. In general, considering growth speed, naturally occurring substances, particularly yeast extracts, are preferable.

The inorganic element source is used to supply phosphoric acid and trace elements. Examples thereof include, in addition to phosphates, inorganic salts (e.g., sulfates, hydrochlorides, nitrates, and phosphates) of metal ions such as sodium, potassium, magnesium, calcium, zinc, manganese, copper, andiron. A required amount of the inorganic element is dissolved in a medium.

In addition, vitamins such as vitamin B₁ or amino acids may be added to the medium.

Further, in accordance with the properties of matsutake fungi to be used, plant extracts, organic acids, nucleic acid-related substances or the like may be added. Examples of the plant extracts include extracts of fruit crops, root crops, and leaf vegetables. Examples of the organic acids include citric acid, tartaric acid, malic acid, fumaric acid, and lactic acid. Examples of the nucleic acid-related substances include commercially available nucleic acids, nucleic acid extracts, yeast, and yeast extracts.

In preparing a solid medium, the amount of carbon source to be used is preferably 10 to 100 g/L, more preferably 10 to 50 g/L, and most preferably 20 to 30 g/L.

The amount of nitrogen source to be used is in nitrogen equivalent, preferably 0.005 to 0.1 mol/L, more preferably 0.007 to 0.07 mol/L, and most preferably 0.01 to 0.05 mol/L.

The amount of phosphate to be used is in phosphorus equivalent, preferably 0.001 to 0.05 mol/L, more preferably 0.005 to 0.03 mol/L, and most preferably 0.01 to 0.02 mol/L. In addition, other inorganic salts, vitamins, plant extracts, organic acids, nucleic acid-related substances, or the like may be optionally added in accordance with the properties of the matsutake fungi. Furthermore, the prepared nutrient substrate solution is adjusted so as to have a pH of preferably 4 to 7, more preferably 4.5 to 6.0, and most preferably 5.0 to 5.5.

<Stationary Liquid Cultivation>

Next, a method for producing matsutake fungi III by stationary cultivation of matsutake fungi II (matsutake fungi cultured or preserved in a solid or liquid medium) in a liquid medium will be described.

Usually, an Erlenmeyer flask with a volume of 100 mL to 2 L is used.

The stationary liquid cultivation starts by inoculating matsutake fungi II on the liquid medium.

The liquid medium is used, in which the ratio (“magnification at the time of inoculation”) of a mixture of the culture liquid containing the matsutake fungi II with a liquid medium to the culture liquid containing the matsutake fungi II is preferably 2:1 to 50:1, and more preferably 3:1 to 30:1.

The culture liquid containing the matsutake fungi II is inoculated on the liquid medium so that the ratio (“concentration of initial mycelia”) between the mass of dried mycelia of matsutake fungi II in the culture liquid containing the matsutake fungi II and the volume of the mixture of the culture liquid containing the matsutake fungi II with the liquid medium becomes preferably 0.05 to 3 g/L, and more preferably 0.1 to 2 g/L.

The temperature for the stationary liquid cultivation is preferably 15 to 30° C., and more preferably 20 to 25° C., and the cultivation period is preferably 30 to 400 days and more preferably 120 to 240 days. If the cultivation period is less than 30 days or more than 400 days, it is difficult to obtain matsutake fungi III having growth ability suitable for mass culture.

In terms of growth ability, the culturing is preferably performed so that the dried mycelia content (unit: g/L) in the culture liquid after the stationary liquid cultivation becomes 2 to 25 times (in a ratio referred to as mycelia increase ratio”) greater than the concentration of initial mycelia.

The liquid medium to be used for the stationary liquid cultivation contains a nutrient substrate so that the medium has an osmotic pressure of preferably 0.01 to 0.8 MPa, more preferably 0.02 to 0.7 MPa, and most preferably 0.03 to 0.5 MPa.

As the nutrient source to be used for the stationary liquid cultivation, the same carbon source, nitrogen source, inorganic element source, vitamins such as vitamin B₁, amino acids, and the like can be used as those used for the solid medium for culturing matsutake fungi I.

The amount of carbon source to be used is preferably 10 to 100 g/L, more preferably 20 to 60 g/L, and most preferably 25 to 45 g/L. Generally, monosaccharides such as glucose are used.

The amount of nitrogen source to be used is in nitrogen equivalent, preferably 0.005 to 0.1 mol/L, more preferably 0.007 to 0.07 mol/L, and most preferably 0.01 to 0.05 mol/L.

When phosphates are used, the amount thereof to be used is in phosphorus equivalent, preferably 0.001 to 0.05 mol/L, more preferably 0.005 to 0.03 mol/L, and most preferably 0.01 to 0.02 mol/L.

In addition, other inorganic salts, vitamins, plant extracts, organic acids, nucleic acid-related substances, or the like may be properly added in accordance with the properties of matsutake fungi.

The prepared nutrient substrate solution has a pH of preferably 4 to 7, more preferably 4.5 to 6.5, and most preferably 5.0 to 6.0.

A part or the whole of the culture liquid containing matsutake fungi III by stationary liquid cultivation may be used again as an inoculation source for stationary liquid cultivation in the stationary liquid cultivation step in the same manner as the culture liquid (or culture product) containing matsutake fungi II.

<Shaking Cultivation>

Next, a method for producing matsutake fungi IV by shaking cultivation of matsutake fungi III will be described.

In general, an Erlenmeyer flask with a volume of 300 mL to 5 L is used.

The shaking cultivation starts by inoculating matsutake fungi III on a liquid medium.

The liquid medium is used, in which the ratio (“magnification at the time of inoculation”) of a mixture of the culture liquid containing the matsutake fungi III with a liquid medium to the culture liquid containing the matsutake fungi III is preferably 2:1 to 50:1, and more preferably 3:1 to 30:1.

Further, in order to secure enough amount of the culture liquid to meet the magnification at the time of inoculation, the stationary liquid culture may be produced using a plurality of culture apparatuses.

The culture liquid containing the matsutake fungi III is inoculated on the liquid medium so that the ratio (“concentration of initial mycelia”) between the mass of dried mycelia of matsutake fungi III in the culture liquid containing the inoculated matsutake fungi III and the volume of the mixture of the culture liquid containing the inoculated matsutake fungi III with the liquid medium becomes preferably 0.05 to 3 g/L, more preferably 0.1 to 2 g/L.

In the shaking cultivation, the temperature is preferably 15 to 30° C. and more preferably 20 to 25° C., and the culture period is preferably 7 to 50 days and more preferably 14 to 28 days.

As power required for the shaking culture, a power of 0.05 to 0.4 kW/m³ for shaking a unit volume of the culture liquid in the Erlenmeyer flask is generally used.

In terms of growth ability, the cultivation is preferably performed so that the dried mycelia content (unit: g/L) in the culture liquid after the stationary liquid cultivation becomes 2 to 25 times (in a ratio referred to as “mycelia increase ratio”) greater than the concentration of initial mycelia.

The liquid medium to be used for the shaking cultivation contains a nutrient substrate so that the medium has an osmotic pressure of preferably 0.01 to 0.8 MPa, more preferably 0.02 to 0.7 MPa, and most preferably 0.03 to 0.5 MPa.

As the nutrient source to be used for the shaking culture, the same carbon source, nitrogen source, inorganic element source, vitamins such as vitamin B₁, amino acids, and the like can be used as those used for the liquid medium for culturing matsutake fungi II.

The amount of carbon source to be used is preferably 10 to 100 g/L, more preferably 20 to 60 g/L, and most preferably 25 to 45 g/L. Generally, monosaccharides such as glucose are used.

The amount of nitrogen source to be used is in nitrogen equivalent, preferably 0.005 to 0.1 mol/L, more preferably 0.007 to 0.07 mol/L, and most preferably 0.01 to 0.05 mol/L.

The amount of phosphate salts to be used is in phosphorus equivalent, preferably 0.001 to 0.05 mol/L, more preferably 0.005 to 0.03 mol/L, and most preferably 0.01 to 0.02 mol/L.

In addition, other inorganic salts, vitamins, amino acids, plant extracts, organic acids, nucleic acid-related substances, or the like may be properly added in accordance with the properties of the matsutake fungi.

The prepared nutrient substrate solution has a pH of preferably 4 to 7, more preferably 4.5 to 6.5, and most preferably 5.0 to 6.0.

<Stirring cultivation>

Next, a method for producing matsutake fungi V, matsutake fungi VI, matsutake fungi VII, and matsutake fungi VIII by stirring cultivation will be described.

The stirring cultivation starts by inoculating matsutake fungi (IV to VII) on a liquid medium.

The liquid medium to be used for the stirring cultivation is prepared in the following manner.

As a nutrient substrate, the same carbon source, nitrogen source, inorganic element source, vitamins such as vitamin B₁, and amino acids may be used as those used for the shaking cultivation.

The amount of carbon source to be used is preferably 10 to 100 g/L, more preferably 20 to 60 g/L, and most preferably 25 to 45 g/L. Starches are preferably used.

When monosaccharides such as glucose, which affects the osmotic pressure of the culture liquid to be stirred, are used in combination, the amount thereof to be used is preferably 0.1 to 60 g/L, more preferably 0.5 to 40 g/L, and most preferably 0.7 to 20 g/L.

The amount of nitrogen source to be used is in nitrogen equivalent, preferably 0.005 to 0.1 mol/L, more preferably 0.007 to 0.07 mol/L, and most preferably 0.01 to 0.05 mol/L.

The amount of phosphates to be used is in phosphorus equivalent, preferably 0.001 to 0.05 mol/L, more preferably 0.005 to 0.03 mol/L, and most preferably 0.01 to 0.02 mol/L.

Further, other inorganic salts, vitamins, amino acids, plant extracts, organic acids, nucleic acid-related substances, and the like may be properly added in accordance with the properties of matsutake fungi.

The pH of the prepared nutrient substrate solution is preferably 4 to 7, more preferably 4.5 to 6.5, and most preferably 5.0 to 6.0.

The liquid medium to be used for stirring cultivation contains a nutrient substrate so that it has an osmotic pressure of preferably 0.01 to 0.8 MPa, more preferably 0.02 to 0.7 MPa, and most preferably 0.03 to 0.5 MPa.

The temperature for the stirring cultivation is 15 to 30° C., preferably 20 to 25° C.

The liquid medium is used, in which the ratio (“magnification at the time of inoculation”) of a mixture of the culture liquid containing the matsutake fungi (IV to VII) with the liquid medium to the culture liquid containing the inoculated matsutake fungi (IV to VII) is preferably 2:1 to 50:1, more preferably 3:1 to 30:1, and most preferably 5:1 to 10:1.

The culture liquid containing the matsutake fungi (IV to VII) is inoculated on the liquid medium so that the volume ratio (“concentration of initial mycelia”) between the mass of dried mycelia of matsutake fungi (IV to VII) in the culture liquid containing inoculated matsutake fungi (IV to VII) and the mixture of the culture liquid containing the inoculated matsutake fungi (IV to VII) with the liquid medium becomes preferably 0.01 to 5 g/L, more preferably 0.05 to 3 g/L, and most preferably 0.1 to 2 g/L.

When matsutake fungi (V to VII) obtained by the stirring culture is used as mother fungi for stirring cultivation, the cultivation period is preferably 3 to 20 days, and particularly preferably 5 to 14 days.

After the cultivation period, the culture liquid contains matsutake fungi (V to VII), which have growth ability suitable for stirring cultivation, at amounts equivalent to dried mycelia content of preferably 0.5 to 10 g/L, more preferably 1 to 8 g/L, and most preferably 1 to 6 g/L.

In terms of growth ability, the culture is preferably performed so that the dried mycelia content (unit: g/L) in the culture liquid after the stationary liquid cultivation becomes 2 to 25 times (in a ratio referred to as “mycelia increase ratio”) greater than the concentration of initial mycelia.

The cultivation period for isolating matsutake mycelia from the matsutake fungi (V to VIII) obtained by the stirring cultivation is 5 to 30 days, more preferably 7 to 20 days, and most preferably 10 to 15 days.

During the above cultivation periods, the time when the assimilation speed of the carbon source decreases remarkably is considered to be the preferable time for terminating the cultivation. However, the time for terminating the cultivation can be properly determined in accordance with production patterns such as production cycle and production cost.

In terms of industrial production, the cultivation is preferably performed so that the dried mycelia content (unit: g/L) in the culture liquid after the stationary liquid cultivation becomes 35 to 100 times (in a ratio referred to as “mycelia increase ratio”) greater than the concentration of initial mycelia.

The culture liquid containing matsutake fungi TV produced by stirring cultivation may be used for a stirring cultivation step with the use of a culture apparatus such as a medium- or large-sized culture tank with a volume of 100 L or more.

The culture apparatus to be used for stirring cultivation is not particularly limited as long as, the apparatus is capable of aeration-cultivation and maintaining sterility. As occasion demands, an apparatus that enables aeration or that can be installed with an aeration apparatus may be used. Therefore, an ordinary small-, medium-, and large-sized culture tank, or a jar fermentor, can be used.

In producing matsutake fungi V by culturing matsutake IV by the use of a jar fermentor or a small-sized culture tank with a volume of less than 100 L, the stirring cultivation is performed preferably without aeration in the liquid medium. The reason is that when the cultivation is performed with aeration in a jar fermentor or small-sized culture tank with a volume of less than 100 L, mycelia grow closely to each other to lose their growing points and their growing ability of mother fungi is damaged.

Further, when the cultivation with deep stirring is performed at industrial scale by the use of a culture apparatus such as a medium- or large-sized culture tank with a volume of 100 L or more, aeration is carried out when needed. In this case, the aeration volume is 0.05 to 1.0 vvm, and in particular preferably 0.2 to 0.5 vvm.

The stirring in the stirring cultivation is controlled by a stirring power required for a unit volume of the culture liquid at an early stage of the cultivation. Generally, by stirring within a power range of preferably 0.01 to 2 kW/m³ and more preferably 0.05 to 1 kW/m³, matsutake mycelia grow favorably. After the early stage, the fungi start to grow, thereby causing insufficient oxygen supply. Further, grown mycelia do not disperse adequately, and thus a larger strength of stirring is properly required. For the deep stirring, preferably, early stage cultivation is conducted with low aeration at low stirring speed and late stage cultivation is performed with high aeration at high stirring speed.

The separation and collection of matsutake mycelia obtained by the deep stirring cultivation may be carried out by conventional methods. Examples of these methods include filtration by a filter press or the like, and centrifugation.

The obtained mycelia are preferably washed well with, for example, distilled water, and then provided for the subsequent hot water extraction step. Further, in order to enhance the extraction efficiency, the mycelia are preferably processed into crushed materials or powders.

[Hot Water Extraction Step]

The hot water used in the hot water extraction step preferably has a temperature of 60 to 100° C., and more preferably 80 to 98° C. It is preferable to carry out the hot water extraction step with stirring or shaking to improve the extraction efficiency. The period for extraction may be properly determined in accordance with, for example, the form of mycelia (e.g., a processed state when they are processed into a crushed or pulverized form), temperature of the hot water, or treatment conditions with or without stirring or shaking, but it is usually about 1 to 6 hours, and preferably about 2 to 3 hours.

After the hot water extraction, a mycelia hot water extract and a mycelia residue can be obtained by an appropriate operation for separation, such as centrifugation or filtration.

[Alkaline Solution Extraction Step]

An alkaline solution to be used in the above alkaline solution extraction step is not particularly limited, but, for example, hydroxides of alkaline metals (sodium, potassium, etc.), in particular an aqueous solution of sodium hydroxide may be used. The alkaline solution preferably has a pH of 8 to 13, and more preferably 9 to 12. The alkaline solution extraction is conducted preferably at a temperature of about 0 to 30° C., and more preferably about 0 to 25° C. A period for extraction may be properly determined in accordance with, for example, the state of the mycelia residue (e.g., a processed state when they are processed into a crushed or pulverized form), a pH value or a temperature of the alkaline solution, or treatment conditions with or without stirring or shaking, but it is usually about 30 minutes to 5 hours, preferably about 1 to 3 hours.

After the alkaline solution extraction, a mycelia residue alkaline solution extract and a mycelia residue can be obtained by an appropriate operation for separation, for example, centrifugation or filtration.

The obtained mycelia residue alkaline solution extract is preferably subjected to neutralization treatment, and used for the subsequent anion exchange resin adsorption step.

[Anion Exchange Resin Adsorption Step]

An extract mixture obtained by mixing the mycelia hot water extract obtained in the hot water extraction step with the mycelia residue alkaline-solution extract obtained in the alkaline solution extract step may be used as it is, namely, in the state containing insolubles, in the subsequent anion exchange resin adsorption step. However, it is preferable to remove the insolubles, or to remove the insolubles and then low molecular weight fractions from the extract mixture for use in the subsequent anion exchange resin adsorption step. For example, the insolubles may be removed by centrifuging the extract mixture containing such insolubles, and only the resultant supernatant may be used in the next anion exchange resin adsorption step. Alternatively, the resultant supernatant obtained by centrifuging the extract mixture containing such insolubles is dialyzed to remove low molecular weight fractions (preferably fractions of low molecular weight substances having a molecular weight of 3500 or less), and the resultant solution may be used in the next anion exchange resin adsorption step.

As an anion exchange resin to be used in the above anion ion exchange resin adsorption step, a publicly known anion exchange resin can be employed. Examples thereof include diethylaminoethyl (DEAE) cellulose and triethylaminoethyl (TEAE) cellulose.

[Elution Step]

An elution solution to be used in the elution step can be properly determined in accordance with the type of an anion exchange resin used in the anion exchange resin adsorption step, and, for example, aqueous sodium chloride solution may be used.

A fraction eluted by the elution step may be used directly as an active ingredient of the infection preventive or therapeutic agent of the present invention. However, the fraction usually contains salts derived from the elution solution, and therefore it is preferable to dialyze the fraction and remove the salts.

The anion exchange resin adsorption fraction of the above extract mixture solution, which is preferably used as an active ingredient of the infection preventive or therapeutic agent and food of the present invention, has the following physicochemical properties.

(1) Carbohydrate content: 60 to 72% (preferably 62 to 70%) in glucose equivalent by a phenol-sulfuric acid method

(2) Protein content: 28 to 40% (preferably 30 to 38%) in albumin equivalent by a copper Folin method

(3) Carbohydrate composition: glucose 61 μg/mg, mannose 3.3 μg/mg, and galactose 2.0 μg/mg

(4) Amino acid composition: aspartic acid and asparagine 10.35 mol %, threonine 5.83 mol %, serine 6.27 mol %, glutamic acid and glutamine 10.49 mol %, glycine 8.55 mol %, alanine 9.19 mol %, valine 6.88 mol %, 1/2-cystine 0.60 mol %, methionine 1.49 mol %, isoleucine 5.36 mol %, leucine 9.25 mol %, tyrosine 2.55 mol %, phenylalanine 4.05 mol %, lysine 5.17 mol %, histidine 2.18 mol %, arginine 4.44 mol %, tryptophan 1.82 mol %, and proline 5.54 mol %

(5) Isoelectric points: An isoelectric point of a main band is around 5.85 by isoelectric focusing.

(6) Nuclear magnetic resonance (NMR)

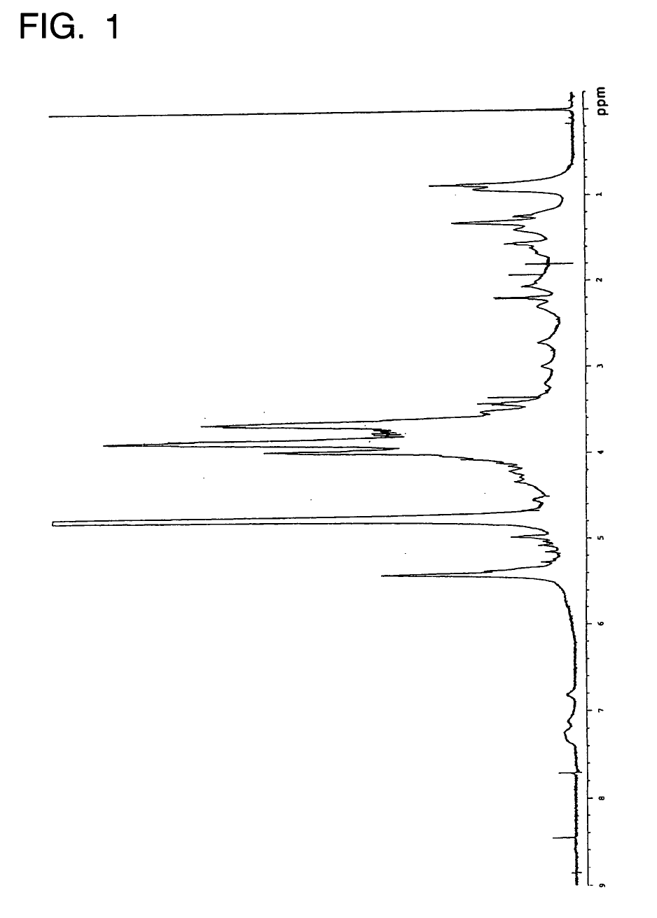

(i) ¹H one-dimensional NMR analysis: a spectrum as shown in FIG. 1 was obtained (refer to Example 6 (6) (i) described below for the measurement conditions).

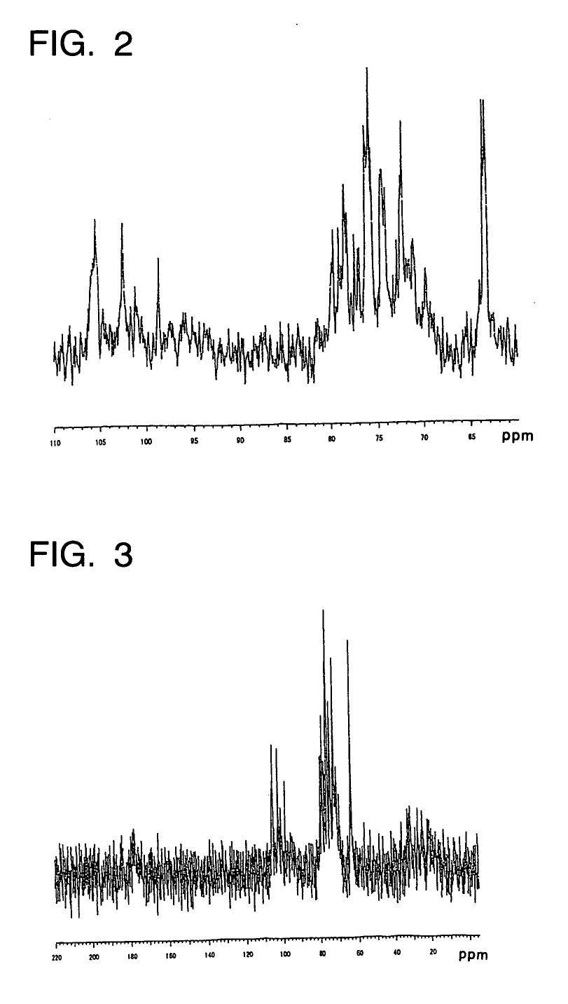

(ii) ¹³C one-dimensional NMR analysis: spectra as shown in FIGS. 2 and 3 were obtained (refer to Example 6 (6) (ii) described below for the measurement conditions).

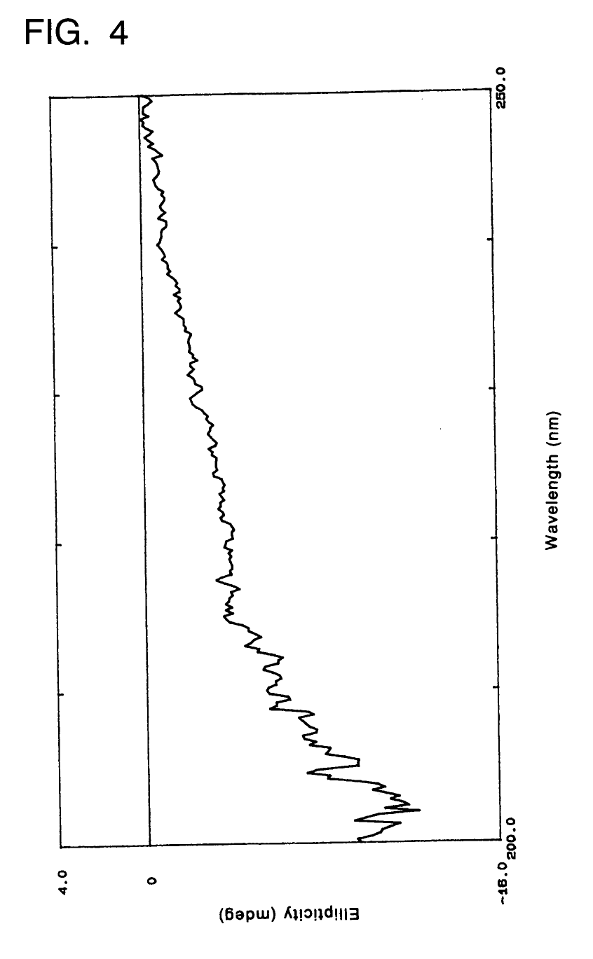

(7) Circular dichroism analysis: a spectrum as shown in FIG. 4 was obtained (refer to Example 6 (7) described below for the measurement conditions).

(8) Optical rotation: 42 (25° C.)

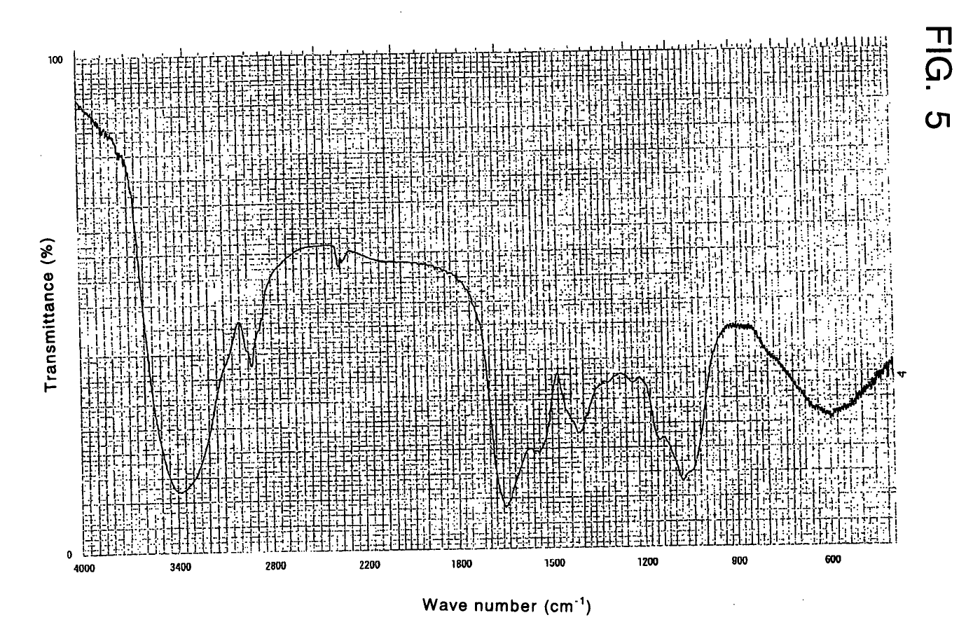

(9) Infrared spectroscopic analysis: a spectrum as shown in FIG. 5 was obtained (refer to Example 6 (9) described below for the measurement conditions).

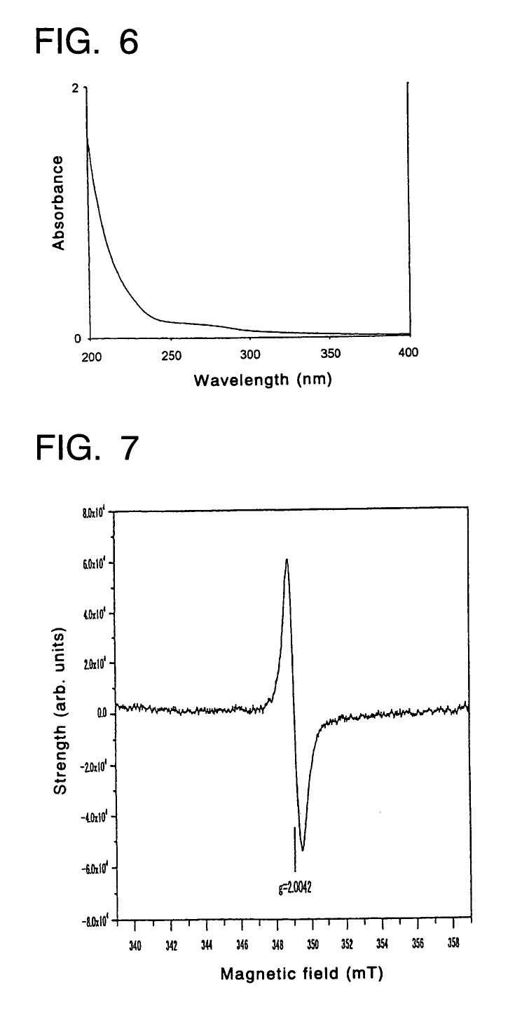

(10) Ultraviolet spectroscopic analysis (UV): a spectrum shown in FIG. 6 was obtained (refer to Example 6 (10) described below for the measurement conditions).

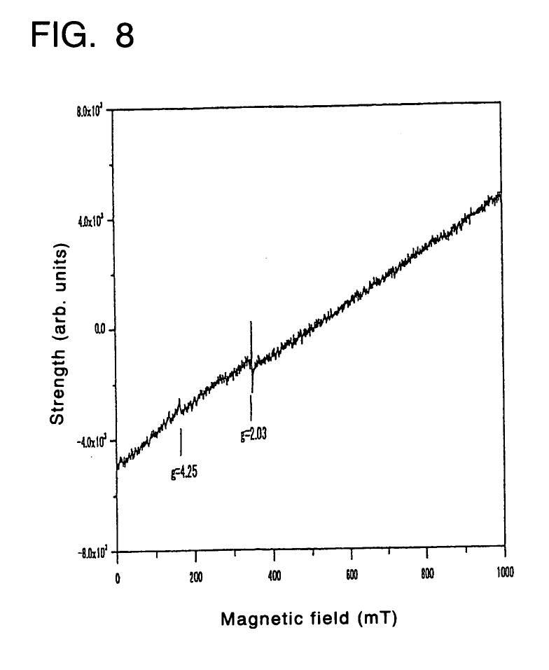

(11) Electron spin resonance (ESR): spectra shown in FIGS. 7 and 8 were obtained (refer to Example 6 (11) described below for the measurement conditions).

(12) Viscosity: reduced viscosity is 108 (30° C.).

(13) Molecular weight: the main component has a molecular weight of 2000 kDa.

(14) Elementary analysis: the contents of carbon (C), hydrogen (H), nitrogen (N), sulfur (S), phosphorus (P), and chlorine (Cl) are 41.3%, 6.0%, 5.1%, 1.0%, 0.052%, and 0.16%, respectively.

(15) α-glucan estimated content: 71% based on the entire carbohydrate

(16) Endotoxin content: 2.5 ng/mg

The active ingredient of the infection preventive or therapeutic agent and food of the present invention is not limited to the above anion exchange resin adsorption fraction, and mycelia, broths, and fruit bodies of the T. matsutake FERM BP-7304 strain, hot water extracts and alkaline extracts thereof, or anion exchange resin adsorption fractions of these extracts may be used as active ingredients.

As mycelia of the T. matsutake FERM BP-7304 strain usable as the active ingredient of the infection preventive or therapeutic agent and food of the present invention, mycelia may be used, for example, in a form obtained directly by removing a medium from a mixture of mycelia obtained by culturing (that is, cultured mycelia) and a medium with an appropriate removing means (e.g., filtration). Alternatively, dried mycelia, which are obtained by removing water from the mycelia after the removal of the medium with an appropriate removing means (e.g., lyophilization) may be used. Further, dried mycelia powders, which are obtained by grinding the above dried mycelia may be used.

As broths of the T. matsutake FERM BP-7304 strain usable as the active ingredient of the infection preventive or therapeutic agent and food of the present invention, a broth may be used, for example, in the form of a mixture of mycelia obtained by cultivation (that is, cultured mycelia) and a medium. Alternatively, a dried broth obtained by removing water from the above mixture with an appropriate removing means (e.g., lyophilization) may be used. Further, dried broth powders, which are obtained by grinding the above dried broth, may be used.

As fruit bodies of the T. matsutake FERM BP-7304 strain usable as the active ingredient of the infection preventive or therapeutic agent and food of the present invention, for example, fruit bodies as they are, or crushed fruit bodies, can be used. Alternatively, dried fruit bodies obtained by removing water therefrom with an appropriate removing means (e.g., lyophilization), may be used. Further, dried fruit body powders obtained by grinding the above dried fruit bodies may be used.

The hot water extract and alkaline extract of the T. matsutake FERM BP-7304 strain, and anion exchange resin adsorption fractions of these extracts, which are usable as the active ingredient of the infection preventive or therapeutic agent and food of the present invention, can be each obtained by methods based on each method described above in the preparation of “anion exchange resin adsorption fractions of mixture solution,” or known methods disclosed in the above-mentioned WO 01/49308 pamphlet. However, the method is not limited thereto.

The infection preventive or therapeutic agent and food of the present invention can be administered to animals, preferably mammals (particularly humans), having as the active ingredient basidiomycetes belonging to the genus Tricholoma or extracts thereof, preferably the T. matsutake FERM BP-7304 strain and extracts thereof, and particularly preferably an M2 fraction thereof, either alone or, if desired, in combination with a pharmaceutically or veterinarily acceptable carrier.

The expression “pathogenic microorganisms” mentioned in the present invention means not only microorganisms themselves having pathogenicity but also causative microorganisms for secondary infection (opportunistic infection) occurring when a host has weak resistance, which are conventionally referred to as nonpathogenic microorganisms or usually harmless microorganisms. It should be noted that the pathogenic “microorganisms” may include everything usually categorized as a so-called microorganism, such as bacteria, viruses, fungi, and protozoans. However, the present invention can exhibit excellent infection preventive or therapeutic effects against, particularly, pathogenic bacteria.

Pathogenic bacteria that are targets of the infection preventive or therapeutic agent and food of the present invention are not particularly limited. Examples thereof include Gram-negative bacteria, such as Pseudomonas aeruginosa, Escherichia coli including 0-157, Helicobacter pylori, Salmonella enteritidis including vancomycin-resistant examples, Neisseria gonorrhoeae, N. meningitides, Haemophilus influenzae, and Enterococcus faecalis including vancomycin-resistant examples; and Gram-positive bacteria, such as Listeria monocytogenes, Mycobacterium tuberculosis, Staphylococcus aureus including methicillin-resistant examples, Streptococcus pyogenes, and Diplococcus pneumoniae. However, the pathogenic bacteria are not limited to these examples.

In the present invention, the expression “infection preventive or therapeutic” means preventing infections with pathogenic microorganisms harmful to various living bodies such as animals and humans (invasion of pathogenic microorganisms into living bodies), inhibiting the onset of the infections (proliferation inhibition), or curing the symptoms (morbidities) caused by pathogenic microorganism infections. For example, infection with Pseudomonas aeruginosa possibly causes septicemia, pneumonia, or the like, and infection with Listeria monocytogenes possibly causes monocytosis. The infection preventive or therapeutic agent and food according to the present invention have effects of preventing subjects from being affected with these infectious diseases, and of curing the diseases they experience when being affected. Therefore, the administration or intake timing of the infection preventive or therapeutic agent and food according to the present invention is not particularly limited. For example, the agent and food can prevent infections when they are administered or taken routinely. In addition, in the case of being affected with the infectious diseases, therapeutic effects can be obtained by promptly administering or taking the agent and food.

The formulation for administration and intake of the infection preventive or therapeutic agent and food of the present invention is not particularly limited to, but may be, for example, oral medicines such as powders, fine particles, granules, tablets, capsules, suspensions, emulsions, syrups, extracts or pills, or parenteral medicines such as injections, liquids for external use, ointments, suppositories, creams for topical application, or eye lotions.

The oral medicines may be prepared by conventional methods using, for example, fillers, binders, disintegrating agents, surfactants, lubricants, flowability-enhancers, diluting agents, preservatives, coloring agents, perfumes, tasting agents, stabilizers, humectants, antiseptics, and antioxidants. Examples of the aforementioned include gelatin, sodium alginate, starch, corn starch, saccharose, lactose, glucose, mannitol, carboxylmethylcellulose, dextrin, polyvinyl pyrrolidone, crystalline cellulose, soybean lecithin, sucrose, fatty acid esters, talc, magnesium stearate, polyethylene glycol, magnesium silicate, silicic anhydride, and synthetic aluminum silicate.

The parenteral administration may take the form of, for example, an injection such as a subcutaneous or intravenous injection, or rectal administration. Among the parenteral formulations, an injection is preferably used.

In preparing injections, for example, water-soluble solvents, such as physiological saline or Ringer's solution, water-insoluble solvents, such as plant oil or fatty acid esters, isotonizing agents such as glucose or sodium chloride, solubilizing agents, stabilizing agents, antiseptics, suspending agents, or emulsifying agents may be optionally used, in addition to the active ingredient.

The infection preventive or therapeutic agent and food of the present invention may be administered in the form of a sustained release preparation using sustained release polymers. For example, the infection preventive or therapeutic agent and food of the present invention may be incorporated in a pellet made of ethylenevinyl acetate polymers, and the pellet may be surgically implanted in a tissue to be treated or which is to be protected from infection.

The infection preventive or therapeutic agent and food of the present invention contain as the active ingredient T. matsutake FERM BP-7304 strain or extracts thereof, anion exchange resin adsorption fractions, or the like in amounts of 0.01 to 99% by mass, and preferably 0.1 to 90% by mass. However, amounts are by no means limited to the aforementioned.

A dose for administration or intake of the infection preventive or therapeutic agent and food of the present invention may be properly determined depending on the kind of disease, the age, sex, body weight, symptoms of a patient, method of administration or intake. The infection preventive or therapeutic agent and food of the present invention may be orally or parenterally administered or taken.

The form of administration or intake is not limited to a medicament, but various forms are available, such as eatable or drinkable products such as health-promoting foods (specified health foods and nutritional-functional foods), as so-called health foods (both including drinkable products), or as feeds. Further, the infection preventive or therapeutic agent and food of the present invention may be administered in the form of an agent that is temporarily kept in the mouth, but then spat out without the retention of most components, for example, a dentifrice, a mouthwash agent, a chewing gum, or a collutorium, or in the form of an inhalant drawn in through the nose. For example, the active ingredient such as T. matsutake FERM BP-7304 strain, extracts thereof, or anion exchange resin adsorption fractions may be added to a desired food (including a drink), a feed, a dentifrice, a mouthwash agent, a chewing gum, a collutorium, or the like as an additive (such as a food additive).

In the above description, the term “specified health food” means a food, for which it is permitted to indicate health functions possessed by that food (permission by Ministry of Health, Labor, and Welfare is required for each food). The term “nutritional-functional food” means a food, for which it is allowed to explicitly state the functions of nutritional components (the standard prescribed by Ministry of Health, Labor, and Welfare should be satisfied). The term “health food” widely means foods in general other than the above-mentioned health-promoting foods, and health food includes health supplements.

Further, antibiotics may be incorporated into the infection preventive or therapeutic agent and food of the present invention to prepare formulations (including food and drink). This allows a dose of the antibiotic to be reduced compared with an ordinary dose for the treatment of infectious diseases, so that the influence of the antibiotic on living bodies can be suppressed. The antibiotics are exemplified by, but not limited to, vancomycin, penicillin, and tetracycline, and may be properly determined in accordance with the kind of relevant infectious disease.

### EXAMPLES

The present invention will be further described by the following Examples, but the technical scope of the present invention is by no means limited by these Examples.

In the following Examples, among pathogenic bacteria, Pseudomonas aeruginosa and Listeria monocytogenes were used as Gram-negative and Gram-positive bacteria, respectively, for evaluation of the ability of T. matsutake to prevent the pathogenic bacteria.

### Example 1

[Preparation of Anion Exchange Resin Adsorption Fraction of Extract Mixture Solution from Mycelia of T. matsutake FERM BP-7304 Strain]

Mycelia of T. matsutake FERM BP-7304 strain were inoculated into a 7-ton culture tank containing 3.5 tons of sterilized medium (3% glucose, 0.3% yeast extract, pH 6.0), and cultured for 4 weeks while being stirred at 25° C. The obtained broth was filtrated with filter cloth, and after mycelia were separated they were washed well with distilled water.

To a portion of the obtained mycelia (about 1 kg), 30 L of purified water was added, and the mixture was stirred for 3 hours in a hot water bath at 98° C. for extraction. After cooling, centrifugation (at 8000 rpm for 30 minutes) was conducted for separation, thereby obtaining a supernatant A₁. 30 L of purified water was added to the residue, and extraction and centrifugation were conducted again under the same conditions as above, thereby obtaining a supernatant A₂.

Subsequently, 20 L of 0.5 mol/L sodium hydroxide aqueous solution was added to the residue after obtaining the supernatant A₂, and the mixture was stirred for 1 hour at 25° C. for extraction. Then centrifugation was conducted, thereby obtaining a supernatant B₁. To the residue, 1.0 mol/L sodium hydroxide aqueous solution was added, and extraction and centrifugation were conducted again under the same conditions as above, thereby obtaining a supernatant B₂. The obtained supernatants B₁ and B₂ were combined, and thereafter the pH of the mixture was adjusted to 7.0 with 1.0 mol/L hydrochloric acid (hereinafter referred to as supernatant B).

A liquid mixture of the supernatants A1, A2, and B (hereinafter referred to as “liquid extract mixture M”) was poured into a dialysis tube (fraction molecular weight of 3500), and dialyzed in flowing water for 48 hours. The inner part of dialyzate was collected and dried with a lyophilizer, thereby obtaining white powder (about 70 g).

A portion (10 g) of the obtained power was dissolved in 500 mL of 50 millimol/L tris-HCl buffer (pH 7.0), and the solution was applied to a column packed with diethylaminoethyl Sephacel (DEAE Sephacel; Pharmacia), which had been equilibrated with the same buffer, thereby obtaining a pass-through fraction (non-adsorption fraction M1). After the column was sufficiently washed with the above tris-HCl buffer, 50 millimol/L tris-HCl buffer (pH 7.0) containing 0.5 mol/L sodium chloride was applied to the column, thereby obtaining an elution fraction (adsorption fraction M2).

The obtained fractions M1 and M2 were each dialyzed at 4° C. for 48 hours with distilled water for injections, and then each inner portion of the dialyzate was lyophilized to obtain powder.

Yields of powder from the fractions M1 and M2 corresponding to mycelia (dried weight) were 7% and 13%, respectively.

### Example 2

[Evaluation of the Preventive Ability of M2 Fraction Regarding Pseudomonas aeruginosa (Single Dose of M2-Fraction One Day Before Inoculation with Pseudomonas aeruginosa)]

(i) Target Bacteria

The Pseudomonas aeruginosa ATCC 27853 strain was used. This strain was maintained at Kitazato Institute Hospital.

(ii) Test Animals

6-week old BALB/c female mice were purchased from Charles River Japan, Inc. The mice were accommodated in polycarbonate cages CL-0103-1 (Clea Japan, Inc.) in a safe and clean rack in an infection experiment animal room, and bred at temperatures of 23±2° C. and humidity of 55±15% under an environment with luminary air flow and with a photoperiod of from 8:00 to 20:00 with free provision of feed CE-2 (Oriental Yeast Co., Ltd.) and sterilized tap water. These mice were quarantined and inspected, and thereafter pre-bred for 1 week (to result in 7-week old mice).

(iii) Preparation of Bacteria Suspension (In Vivo Passage)

The Pseudomonas aeruginosa ATCC 27853 strain, which had been frozen-stored in an ultra low freezer, was added to 1 mL of Heart Infusion Broth (HI liquid medium) to be resuspended. The resuspended strain was streaked on a plate medium (HIA medium) prepared by adding 1.5% agarose to an HI liquid medium using a platinum loop, and cultured in an incubator at 37° C. for 18 hours. The bacteria were picked up (fished) from a single colony grown on the HIA medium with a platinum loop, and streaked and cultured on an HIA medium. A colony of the bacteria grown on the HIA medium was scraped and picked up with a platinum loop. The colony was diluted with PBS to have a concentration of 108 CFU/mL, and 200 μL of the diluted colony was inoculated into a BALB/c mouse via the tail vein.

After 48 hours, the spleen of the mouse was extirpated and ground with a blender. The ground spleen was resuspended in 5 mL of PBS to prepare a bacterial suspension. The prepared bacterial suspension was intraperitoneally inoculated into a BALB/c mouse. After 48 hours, the spleen of the mouse was extirpated and ground with a blender in the same manner as above. Then, the ground spleen was resuspended in 5 mL PBS. The resuspended spleen was streaked on an HIA medium with a platinum loop and cultured in an incubator at 37° C. for 18 hours.

Bacteria were picked up from a single colony grown on the HIA medium with a platinum needle and resuspended in 5 mL of HI liquid medium. The resuspended bacteria were subjected to shaking cultivation in a thermostat bath at 37° C. for 3 hours. After the culture, a bacterial suspension was added into 300 mL of HI medium and subjected to shaking-mixing cultivation in a thermostat bath at 37° C. for further 3 hours.

After the culture, the bacterial suspension was centrifuged at high speed of 5000×g at 4° C. for 30 minutes and a supernatant was removed. The suspension was resuspended in 20 mL of HI medium containing 50% glycerol, dispensed at 1 mL at a time, and stored at −80° C. until use in the experiment. A portion of the bacterial suspension was used to measure the number of bacteria by a 10-times serial dilution method.

(iv) Inoculation of Bacteria and Administration of Test Substance

The 7-week old mice as described in (ii) above were treated 1 day before the inoculation with the target bacteria with a single intraperitoneal administration of the M2 fraction (test substance) obtained in Example 1. The concentration of the test substance per administration was determined to be 100 mg/kg, 20 mg/kg, or 5 mg/kg based on the weight of each mouse measured at the time of administration, and then the intraperitoneal administration was conducted.

One day following the administration of the test substance, 5.0×10⁶ CFU of Pseudomonas aeruginosa prepared in (iii) above was intraperitoneally inoculated. To control groups, physiological saline was intraperitoneally administered one day before the bacteria inoculation.

(v) Measurement of Survival Rate

For the calculation of survival rate, 8 mice for one group were used. During the 2 weeks following the bacteria inoculation, the viability of the mice was daily observed. The survival rate was defined as a value obtained by dividing the number of living individuals during the observation by the total number of individual per group, and multiplying the resultant value by 100.

(vi) Statistical Analysis

Significant differences between the control group and each group treated with the test substance were evaluated by a Mann-Whitney U test, and significant differences between the treated groups were evaluated by a Kruskal-Wallis H test. For each test, less than 5% was defined to constitute a significance level.

(vii) Effect of Infection Resistance in the Case of Single Administration of M2 Fraction 1 Day Before Inoculation with Pseudomonas aeruginosa

BALB/c mice that had been treated with an administration of the M2 fraction 1 day before the bacteria inoculation were intraperitoneally inoculated with a lethal dose of the Pseudomonas aeruginosa ATCC 27853 strain at a concentration of 5.0×10⁶ CFU, and the survival rates were calculated in terms of the infection preventive ability of the M2 fraction. The results are shown in Table 1.

TABLE 1

| | Single administration of M2 fraction 1 day before inoculation with Pseudomonas aeruginosa | | | | | | | | | | | | | |
|---|---|---|---|---|---|---|---|---|---|---|---|---|---|---|
| | (survival rate over the period from the 1st day to the 14th day following inoculation) | | | | | | | | | | | | | |
| | 1st | 2nd | 3rd | 4th | 5th | 6th | 7th | 8th | 9th | 10th | 11th | 12th | 13th | 14th |
| | day | day | day | day | day | day | day | day | day | day | day | day | day | day |
| PBS | 8/8 | 6/8 | 6/8 | 6/8 | 2/8 | 2/8 | 2/8 | 2/8 | 2/8 | 2/8 | 2/8 | 2/8 | 2/8 | 2/8 |
| | 100 | 75 | 75 | 75 | 25 | 25 | 25 | 25 | 25 | 25 | 25 | 25 | 25 | 25 |
| 100 | 8/8 | 8/8 | 8/8 | 8/8 | 8/8 | 8/8 | 8/8 | 8/8 | 8/8 | 8/8 | 8/8 | 8/8 | 8/8 | 8/8 |
| mg/kg | 100 | 100 | 100 | 100 | 100 | 100 | 100 | 100 | 100 | 100 | 100 | 100 | 100 | 100 |
| 20 | 8/8 | 8/8 | 8/8 | 8/8 | 8/8 | 8/8 | 8/8 | 8/8 | 8/8 | 8/8 | 8/8 | 8/8 | 8/8 | 8/8 |
| mg/kg | 100 | 100 | 100 | 100 | 100 | 100 | 100 | 100 | 100 | 100 | 100 | 100 | 100 | 100 |
| 5 | 7/8 | 6/8 | 6/8 | 6/8 | 6/8 | 6/8 | 6/8 | 6/8 | 6/8 | 6/8 | 6/8 | 6/8 | 6/8 | 6/8 |
| mg/kg | 87.5 | 75 | 75 | 75 | 75 | 75 | 75 | 75 | 75 | 75 | 75 | 75 | 75 | 75 |

upper boxes: (number of living individuals)/(total number of treated individuals)
lower boxes: survival rate (%)

As shown in Table 1, dead individuals were found in the PBS-treated group as a control group on the 2nd day following the bacteria inoculation. 2 dead individuals were found on the 2nd day and 4 dead individuals were found on the 5th day following the bacteria inoculation, and the final survival rate at the end of the 2-week experiment was 25%. Among the M2 fraction-treated groups, all individuals of the groups treated with 100 mg/kg and 20 mg/kg remained alive until the completion of the experiment from the 1st day following the bacteria inoculation, so the survival rates were 100%. In the group treated with 5 mg/kg, 1 individual was found dead on the 1st day and another 1 individual was found dead on the 2nd day following the bacteria inoculation, and the final survival rate was 75%.

The survival period until the 14th day of each group was assayed using a Mann-Whitney U test. In comparison with the PBS control group, the groups treated with 100 mg/kg and 20 mg/kg of the M2 fraction were found to exhibit a significant extension of the survival period (P<0.05). Among the M2 treated-groups, no significant difference was found.

### Example 3

[Evaluation of Preventive Ability of M2 Fraction Regarding Pseudomonas aeruginosa (Single Dose of M2 Fraction 3 Days Before Inoculation with Pseudomonas aeruginosa)]

The evaluation tests were conducted in the same manner as Example 2 except that the M2 fraction was administered 3 days before the inoculation with Pseudomonas aeruginosa instead of 1 day before. The results are shown in Table 2.

TABLE 2

| | Single administration of M2 fraction 3 days before inoculation with Pseudomonas aeruginosa | | | | | | | | | | | | | |
|---|---|---|---|---|---|---|---|---|---|---|---|---|---|---|
| | (survival rate over the period from the 1st day to the 14th day following inoculation) | | | | | | | | | | | | | |
| | 1st | 2nd | 3rd | 4th | 5th | 6th | 7th | 8th | 9th | 10th | 11th | 12th | 13th | 14th |
| | day | day | day | day | day | day | day | day | day | day | day | day | day | day |
| PBS | 8/8 | 6/8 | 6/8 | 6/8 | 2/8 | 2/8 | 2/8 | 2/8 | 2/8 | 2/8 | 2/8 | 2/8 | 2/8 | 2/8 |
| | 100 | 75 | 75 | 75 | 25 | 25 | 25 | 25 | 25 | 25 | 25 | 25 | 25 | 25 |
| 100 | 8/8 | 8/8 | 8/8 | 8/8 | 8/8 | 8/8 | 8/8 | 8/8 | 8/8 | 8/8 | 8/8 | 8/8 | 8/8 | 8/8 |
| mg/kg | 100 | 100 | 100 | 100 | 100 | 100 | 100 | 100 | 100 | 100 | 100 | 100 | 100 | 100 |
| 20 | 8/8 | 7/8 | 7/8 | 7/8 | 7/8 | 7/8 | 7/8 | 7/8 | 7/8 | 7/8 | 7/8 | 7/8 | 7/8 | 7/8 |
| mg/kg | 100 | 87.5 | | | | | | | | | | | | |
| 5 | 8/8 | 6/8 | 4/8 | 4/8 | 4/8 | 4/8 | 4/8 | 4/8 | 4/8 | 4/8 | 4/8 | 4/8 | 4/8 | 4/8 |
| mg/kg | 100 | 75 | 50 | 50 | 50 | 50 | 50 | 50 | 50 | 50 | 50 | 50 | 50 | 50 |

upper boxes: (number of living individuals)/(total number of treated individuals)
lower boxes: survival rate (%)

As shown in Table 2, dead individuals were found in the PBS-treated group as a control group since the 2nd day following the bacteria inoculation. 2 dead individuals were found on the 2nd day and 4 dead individuals were found on the 5th day following the bacteria inoculation, and the final survival rate at the end of the 2-week experiment was 25%. Among the M2 fraction-treated groups, all individuals of the group treated with 100 mg/kg remained alive until the completion of the experiment from the 1st day following the bacteria inoculation, so the survival rate was 100%. In the group treated with 20 mg/kg, only 1 dead individual was found on the 2nd day following the bacteria inoculation, and the final survival rate was 87.5%. In the group treated with 5 mg/kg, 2 dead individuals were found on the 2nd day and 2 dead individuals were found on the 3rd day following the bacteria inoculation, and the final survival rate was 50%.

The survival period until the 14th day of each group was assayed using a Mann-Whitney U test. In comparison with the PBS control group, only the treated group with 100 mg/kg of the M2 fraction was found to exhibit a significant extension of the survival period (P<0.05). Among the M2 treated-groups, no significant difference was found.

### Example 4

[Evaluation of Infection Preventive Ability of M2 Fraction Regarding Listeria monocytogenes (Single Administration of M2 Fraction 1 Day Before Inoculation with Listeria monocytogenes)]

(i) Target Bacteria

The Listeria monocytogenes EGD strain was used. This strain was maintained at Kitazato Institute Hospital.

(ii) Test Animals

6-week old C57BL/6 female mice were purchased from Charles River Japan, Inc. The mice were accommodated in polycarbonate cages CL-0103-1 (Clea Japan, Inc.) in a safe and clean rack in an infection test animal room, and bred at temperatures of 23±2° C. and humidity of 55±15% under an environment with luminary air flow and with a photoperiod of from 8:00 to 20:00 with free provision of feed CE-2 (Oriental Yeast Co., Ltd.) and sterilized tap water. These mice were quarantined and inspected, and thereafter pre-bred for 1 week (to result in 7-week old mice).

(iii) Preparation of Bacteria Suspension (In Vivo Passage)

The Listeria monocytogenes EGD strain, which had been frozen-stored in an ultra low freezer, was added to 1 mL of Tryptic Soy Broth (TS liquid medium) containing 1% dextrose to be resuspended. The resuspended strain was streaked using a platinum loop on a plate medium (TSA medium) prepared by adding 1.5% agarose to a TS liquid medium, and cultured in an incubator at 37° C. for 18 hours. The bacteria were picked up from a single colony grown on the TSA medium with a platinum loop, and dissolved in 10 mL of TSA liquid medium. Then, the solution was subjected to shaking-cultivation in a thermostat bath at 37° C. for 3 hours. After the cultivation, the bacterial suspension was centrifuged at a high speed of 5000×g at 4° C. for 30 minutes. After a supernatant was removed, 10 mL of PBS was added to the suspension for resuspension, thereby preparing a bacterial suspension.

200 μL of the prepared bacterial suspension was intraperitoneally inoculated into a C57BL/6 mouse. After 48 hours, the spleen of the mouse was extirpated and ground with a blender. The ground spleen was resuspended in 10 mL of PBS. The resultant suspension was streaked on a TSA medium using a platinum loop, and cultured in an incubator at 37° C. for 18 hours. 200 μl of the prepared bacterial suspension was intraperitoneally inoculated into a C57BL/6 mouse. After 48 hours, the spleen of the mouse was extirpated and ground with a blender in the same manner as above. The ground spleen was resuspended in 10 mL of PBS. The resultant suspension was streaked on a TSA medium with a platinum loop and cultured in an incubator at 37° C. for 18 hours.

Bacteria were picked up from a single colony grown on the TSA medium with a platinum loop and dissolved in 10 mL of HI liquid medium. Then, the bacterial suspension was subjected to shaking-cultivation in a thermostat bath at 37° C. for 3 hours. After the cultivation, the bacterial suspension was added to 300 mL of HI medium and subjected to shaking-mixing cultivation in a thermostat bath at 37° C. for further 3 hours.

After the cultivation, the resultant bacterial suspension was centrifuged at a high speed of 5000×g at 4° C. for 30 minutes and a supernatant was removed. The resultant product was resuspended in 20 mL of TS medium containing 50% glycerol, dispensed at 1 mL at a time, and stored −80° C. until use in the experiment. A part of the bacterial suspension was used to measure the number of bacteria by a 10-times serial dilution method.

(iv) Inoculation of Bacteria and Administration of Test Substance

The 7-week old mice as described in (ii) above were treated 1 day before the inoculation with the target bacteria with a single intraperitoneal administration of the M2 fraction (test substance) obtained in Example 1. The concentration of the test substance per administration was determined to be 100 mg/kg, 20 mg/kg, and 5 mg/kg based on the weight of each mouse measured at the time administration, and then the intraperitoneal administration was conducted.

One day following the administration of the test substance, 1.0×10⁶ CFU of Listeria monocytogenes prepared in (iii) above was intraperitoneally inoculated. To a control group, physiological saline was intraperitoneally administered 1 day before the bacterial inoculation.

(v) Measurement of Survival Rate

For the calculation of survival rate, 8 mice were used for 1 group. During the 2 weeks following the bacterial inoculation, the viability of the mice was daily observed. The survival rate was defined as a value obtained by dividing the number of living individuals during the observation by the total number of individuals per group and multiplying the resultant value by 100.

(vi) Statistical Analysis

Significant differences between the control group and each group treated with the test substance were evaluated by a Mann-Whitney U test, and significant differences between the treated groups were evaluated by a Kruskal-Wallis H test. For each test, less than 5% was defined to constitute a significance level.

(vii) Effect of Infection Resistance in the Case of Single Administration of M2 Fraction 1 Day Before Inoculation with Listeria monocytogenes

C57BL/6 mice that had been treated with the administration of the M2 fraction 1 day before the bacteria inoculation were intraperitoneally inoculated with a lethal dose of Listeria monocytogenes EGD strain at a concentration of 1.0×10⁶ CFU, and the survival rates were calculated in terms of the infection preventive ability of the M2 fraction. The results are shown in Table 3.

TABLE 3

| | Single administration of M2 fraction 1 day before inoculation with Listeria monocytogenes | | | | | | | | | | | | | |
|---|---|---|---|---|---|---|---|---|---|---|---|---|---|---|
| | (survival rate over the period from the 1st day to the 14th day following inoculation) | | | | | | | | | | | | | |
| | 1st | 2nd | 3rd | 4th | 5th | 6th | 7th | 8th | 9th | 10th | 11th | 12th | 13th | 14th |
| | day | day | day | day | day | day | day | day | day | day | day | day | day | day |
| PBS | 8/8 | 8/8 | 6/8 | 2/8 | 2/8 | 2/8 | 2/8 | 2/8 | 2/8 | 2/8 | 2/8 | 2/8 | 2/8 | 2/8 |
| | 100 | 100 | 75 | 25 | 25 | 25 | 25 | 25 | 25 | 25 | 25 | 25 | 25 | 25 |
| 100 | 8/8 | 8/8 | 8/8 | 8/8 | 8/8 | 8/8 | 8/8 | 8/8 | 8/8 | 8/8 | 8/8 | 8/8 | 8/8 | 8/8 |
| mg/kg | 100 | 100 | 100 | 100 | 100 | 100 | 100 | 100 | 100 | 100 | 100 | 100 | 100 | 100 |
| 20 | 8/8 | 8/8 | 8/8 | 8/8 | 8/8 | 8/8 | 8/8 | 8/8 | 8/8 | 8/8 | 8/8 | 8/8 | 8/8 | 8/8 |
| mg/kg | 100 | 100 | 100 | 100 | 100 | 100 | 100 | 100 | 100 | 100 | 100 | 100 | 100 | 100 |
| 5 | 8/8 | 8/8 | 8/8 | 8/8 | 7/8 | 7/8 | 7/8 | 7/8 | 7/8 | 7/8 | 7/8 | 7/8 | 7/8 | 7/8 |

| | | | | | |
|---|---|---|---|---|---|
| mg/kg | 100 | 100 | 100 | 100 | 87.5 |

upper boxes: (number of living individuals)/(total number of treated individuals)
lower boxes: survival rate (%)

As shown in Table 3, dead individuals were found in the PBS-treated group as a control group since the 3rd day following the bacteria inoculation. 2 dead individuals were found on the 3rd day and 4 dead individuals were found on the 4th day following the bacteria inoculation, and the final survival rate at the end of the 2-week experiment was 25%. Among the M2 fraction-treated groups, all individuals of the groups treated with 100 mg/kg and 20 mg/kg remained alive until the completion of the experiment from the 1st day following the bacteria inoculation, and the survival rates were 100%. In the group treated with 5 mg/kg, 1 dead individual was found on the 5th day following the bacteria inoculation, and the final survival rate was 87.5%.

Further, the survival period until the 14th day of each group was assayed using a Mann-Whitney U test. In comparison with the PBS-treated control group, the groups treated with 100 mg/kg, 20 mg/kg, and 5 mg/kg of the M2 fraction were found to exhibit a significant extension of the survival period (P<0.05). Among the M2 fraction-treated groups, no significant difference was found.

### Example 5

[Evaluation of Preventive Ability of M2 Fraction Regarding Listeria monocytogenes (Single Administration of M2 Fraction 3 Days Before Inoculation with Listeria monocytogenes)]

The evaluation tests were conducted in the same manner as Example 4 except that the M2 fraction was administered 3 days before the inoculation with Listeria monocytogenes instead of 1 day before. The results are shown in Table 4.

TABLE 4

| | Single administration of M2 fraction 3 day before inoculation with Listeria monocytogenes | | | | | | | | | | | | | |
|---|---|---|---|---|---|---|---|---|---|---|---|---|---|---|
| | (survival rate over the period from the 1st day to the 14th day following inoculation) | | | | | | | | | | | | | |
| | 1st | 2nd | 3rd | 4th | 5th | 6th | 7th | 8th | 9th | 10th | 11th | 12th | 13th | 14th |
| | day | day | day | day | day | day | day | day | day | day | day | day | day | day |
| PBS | 8/8 | 8/8 | 6/8 | 2/8 | 2/8 | 2/8 | 2/8 | 2/8 | 2/8 | 2/8 | 2/8 | 2/8 | 2/8 | 2/8 |
| | 100 | 100 | 75 | 25 | 25 | 25 | 25 | 25 | 25 | 25 | 25 | 25 | 25 | 25 |
| 100 | 8/8 | 8/8 | 8/8 | 8/8 | 8/8 | 8/8 | 8/8 | 8/8 | 8/8 | 8/8 | 8/8 | 8/8 | 8/8 | 8/8 |
| mg/kg | 100 | 100 | 100 | 100 | 100 | 100 | 100 | 100 | 100 | 100 | 100 | 100 | 100 | 100 |
| 20 | 8/8 | 8/8 | 8/8 | 8/8 | 8/8 | 8/8 | 8/8 | 8/8 | 8/8 | 8/8 | 8/8 | 8/8 | 8/8 | 8/8 |
| mg/kg | 100 | 100 | 100 | 100 | 100 | 100 | 100 | 100 | 100 | 100 | 100 | 100 | 100 | 100 |
| 5 | 8/8 | 8/8 | 8/8 | 7/8 | 7/8 | 7/8 | 7/8 | 7/8 | 7/8 | 7/8 | 7/8 | 7/8 | 7/8 | 7/8 |

| | | | | |
|---|---|---|---|---|
| mg/kg | 100 | 100 | 100 | 87.5 |

upper boxes: (number of living individuals)/(total number of treated individuals)
lower boxes: survival rate (%)

As shown in Table 4, dead individuals were found in the PBS-treated group as a control group since the 3rd day following the bacterial inoculation. 2 dead individuals were found on the 3rd day and 4 dead individuals were found on the 4th day following the bacteria inoculation, and the final survival rate at the end of the 2-week experiment was 25%. Among the M2 fraction-treated groups, all individuals of the groups treated with 100 mg/kg and 20 mg/kg remained alive until the completion of the experiment from the 1st day following the bacteria inoculation, so the survival rates were 100%. In the group treated with 5 mg/kg, 1 dead individual was found on the 4th day following the bacteria inoculation, and the final survival rate was 87.5%.

Further, the survival period until the 14th day of each group was assayed using a Mann-Whitney U test. In comparison with the PBS-treated control group, the groups treated with 100 mg/kg, 20 mg/kg, and 5 mg/kg of the M2 fraction were found to exhibit a significant extension of the survival period (P<0.05). Among the M2 fraction-treated groups, no significant difference was found.

[Consideration]

As defensive responsive mechanisms of the living body at early stages of Pseudomonas aeruginosa infection, bacteria are eliminated mainly by neutrophils or macrophages. However, as defensive responses to a large amount of bacteria that cannot be treated by short-term activities of these phagocytes, adaptive immunity (antigen-specific antibodies particularly in the case of Pseudomonas aeruginosa) that begins to take place a few days following infection eliminates bacteria. According to the results of the above Examples 2 and 3, a single administration of the M2 fraction cannot cause long-lasting resistance to Pseudomonas aeruginosa infection, which is effected by immunostimulation in the living body after the M2 fraction administration. However, there is a possibility that administration for consecutive days brings about the effects.

On the other hand, Listeria monocytogenes, like bacteria such as Mycobacterium tuberculosis and Salmonella, is a typical intracellular parasitic bacterium, which parasitizes and multiplies in a cell. Listeria monocytogenes can escape from phagocytosis via macrophages and is capable of intracytoplasimc multiplication. Therefore, antigen-specifically sensitized T cells and macrophages activated by cytokine are main bodies as infection defense to eliminate the bacteria. Infection defense by antibodies or complements is ineffective. The results of the above Examples 4 and 5 show the possibility that the M2 fraction differentiates and induces Th1 cells to cause strong defensive immunity.

### Example 6

[Examination of the Physicochemical Properties of the M2 Fraction]

The M2 fraction obtained in Example 1 and an m2 fraction derived from fruit bodies of commercially available matsutake, which is obtained in Example 7 described below, were examined in terms of physicochemical properties. Measurement methods and the results thereof will be described below.

(1) Determination of Carbohydrates

Carbohydrate content was determined by colorimetry using a phenol-sulfuric acid method. The content of carbohydrates in the M2 fraction was 62% in glucose equivalent.

The operation of Example 1 was separately conducted twice, and likewise the carbohydrate contents in the 2 kinds of obtained M2 fractions were determined by colorimetry using a phenol-sulfuric acid method. Their carbohydrate contents were 69% and 70% in glucose equivalent, respectively.

The content of carbohydrates in the m2 fraction was 35% in glucose equivalent.

Further, iodine color reaction was conducted, and both the M2 fraction and the m2 fraction exhibited negative reactions. Thus, it was considered that carbohydrates having different properties from starch were present therein.

(2) Determination of Proteins

Protein content was determined by colorimetry using a copper-Folin method. The content of proteins in the M2 fraction was 38% in albumin equivalent.

The operation of Example 1 was separately conducted twice, and likewise the protein contents in the 2 kinds of obtained M2 fractions were determined by colorimetry using a copper-Folin method. Their protein contents were 31% and 30% in albumin equivalent, respectively.

The content of proteins in the m2 fraction was 65% in albumin equivalent.

(3) Analysis of Carbohydrate Composition

Into a tube, 1.0 mg of the M2 fraction and 0.2 mL of 2 mol/L trifluoroacetic acid were charged, and hydrolyzed at 100° C. for 6 hours. The reaction mixture was dried under reduced pressure by an evaporator to obtain a residue. The residue was dissolved in 500 μL of pure water, and further diluted to a double volume or a ten-fold volume with pure water. To 50 μL of the diluted solution, 500 ng of heptose was added as an internal standard substance, and the solution was applied to a high performance liquid chromatograph LC-9A (Shimadzu Corp.) equipped with a column TSK-gel Sugar AXGLC-9A (15 cm×4.6 mm I.D.; Tosoh Corporation.) and a spectrophotometer RF-535 (Shimadzu Corp.) as a detector. The column temperature was 70° C. As a mobile phase, 0.5 M potassium borate buffer (pH 8.7) was used at a flow rate of 0.4 mL/min. For the conditions of post-column labeling, 1% arginine/3% boric acid was used as a reaction reagent, at a flow rate of 0.5 mL/min. The reaction temperature was 150° C., and the wavelengths for detection were EX 320 nm and EM 430 nm.

The carbohydrate composition of the M2 fraction was as follows in the order of descending content: glucose 61 μg/mg, mannose 3.3 μg/mg, and galactose 2.0 μg/mg.

In addition, the carbohydrate composition of the m2 fraction was as follows in the order of descending content: glucose 12.9 μg/mg, galactose 12.6 μg/mg, mannose 5.6 μg/mg, fucose 3.5 μg/mg, and xylose 0.4 μg/mg.

(4) Analysis of Amino Acid Composition

Acid hydrolysis was conducted in the following manner. 0.33 mg of the M2 fraction and 0.2 mL of 6 mol/L hydrochloric acid were charged into a tube, and hydrolyzed at 110° C. for 22 hours. Then, the reaction mixture was dried under reduced pressured by an evaporator to obtain a residue. The residue was dissolved in 0.5 mL of pure water, and 50 μL thereof was used for amino acid analysis.

In addition, alkaline hydrolysis (for tryptophan analysis) was conducted in the following manner. 0.48 mg of the M2 fraction was charged into a plastic tube, and 100 μL of 1% n-octyl alcohol-4.2 mol/L sodium hydroxide solution containing 5 mg of soluble starch was added thereto. This plastic tube was placed in a glass test tube to create a sealed and vacuum condition, and hydrolyzed at 110° C. for 16 hours. After being cooled by air, the sealing was broken and the plastic tube was cooled in ice. Then, 1.0 mol/L of hydrochloric acid was added to neutralize the reaction solution. Further, 840 μL of purified water was added so that the solution had the total volume of 1000 μL, and 50 μL thereof was used for amino acid analysis.

The quantitative determination was conducted by ninhydrin colorimetry using a amino acid analyzer L-8500 (Hitachi, Ltd.) as equipment.

The amino acid composition was as follows: aspartic acid and asparagine 10.35 mol %, threonine 5.83 mol %, serine 6.27 mol %, glutamic acid and glutamine 10.49 mol %, glycine 8.55 mol %, alanine 9.19 mol %, valine 6.88 mol %, 1/2-cystine 0.60 mol %, methionine 1.49 mol %, isoleucine 5.36 mol %, leucine 9.25 mol %, tyrosine 2.55 mol %, phenylalanine 4.05 mol %, lysine 5.17 mol %, histidine 2.18 mol. %, arginine 4.44 mol %, tryptophan 1.82 mol %, and proline 5.54 mol %.

(5) Analysis of Isoelectric Point

The M2 fraction was adjusted to be 1 mg/mL. To (i) a solution prepared by adding 10 μL of pure water to 10 μL of the M2 fraction solution or (ii) 20 μL of the M2 fraction solution (equivalent to approximately 1.14 μg of protein), saccharose was added so that the concentration thereof became approximately 40% (volume/volume), and then electrophoresis was performed. The conditions of the electrophoresis were as follows.

Gel: IEF-PAGEmini (4%, pH 3 to 10; Tefco)

Buffer for electrophoresis: (cathode) 0.04 mol/L sodium hydroxide solution, (anode) 0.01 mol/L phosphate solution

Conditions for electrophoresis: electrophoresis was preformed at 100 V for 30 minutes, then at 300 V for 20 minutes, and further at 500 V for 40 minutes

pI marker: each band was 1.35 g (Pharmacia)

Staining: silver staining

The isoelectric point of a main band was around 5.85.

(6) Nuclear Magnetic Resonance Analysis (NMR)

The conditions for measurements were as follows.

(i) ¹H One-Dimensional NMR Measurement

To 7 mg of the M2 fraction, 800 μL of D₂O was added, and dissolution by ultrasound was attempted for about 5 minutes. Thereafter, the resultant solution was centrifuged and a supernatant thereof was used for measurement. “The conditions for measurement were as follows.

Measurement instrument: UNITY INOVA 600 (Varian)

Observation frequency: 599.6 MHz (1H nuclear)

Solvent: D₂O solution (saturated solution)

Standard: TSP 0.00 ppm (¹H)

Temperature: 25° C.

Period for repeating: 7.0 seconds (¹H)

Number of accumulation: 256

The obtained spectrum is shown in FIG. 1. Strong signals derived from carbohydrates were observed in the range from 3.0 to 5.6 ppm. When signals observed in the range from 0.5 to 3.0 ppm were considered to be derived from side chains of amino acids, the signal strength from carbohydrates are much stronger than the signal strength from amino acids. Thus, the M2 fraction was assumed to contain numerous carbohydrates in its structure. In addition, NMR signals of aromatic amino acids were observed in the range from 6.6 to 7.6 ppm.

Further, the estimated content of α-glucan was 71%.

(ii) ¹³C One-Dimensional NMR Measurement

The M2 fraction was dissolved in D₂O/CD₃OD (725/25) so as to have a concentration of approximately 20.5 mg/0.75 mL, and measurement was conducted under the following operation conditions.

Observation frequency: 125.8 MHz

Standard: deuterated methanol (a=49 ppm)

Temperature: 45° C.

Observation width: 31.4 kHz

Data point: 64 K

Pulse width: approximately 41°

Period for pulse repetition: 2.5 seconds

Number of accumulation: 4000

Decoupling: ¹H complete decoupling

The results are shown in FIGS. 2 and 3. Signals derived from carbohydrates and amino acids were observed, and the signal strength from carbohydrates was stronger than that from amino acids. Since many component carbohydrates of the M2 fraction were glucose, it was assumed that signals in the range from 95 to 110 ppm were from a carbon at the 1-position of glucose, signals in the range around 10.5 ppm were from a carbon at the P1-position, and signals around 102 ppm and 99 ppm were from a carbon at the α1-position. According to these results, at least 3 kinds of bonding types were presumed. Though signals around 63 ppm were from a carbon at the 6-position, 3 kinds of signals were herein present and this supported the conclusion that the M2 fraction has 3 or more kinds of bonding types. Further, according to signals around 70 to 80 ppm, a carbon at the 4-position was considered to be involved in the bonding, and thus it was presumed that α1-4 bonding and β1-4 bonding were present.

(7) Circular Dichroism Analysis (CD)

Water was added to approximately 3 mg of the M2 fraction so as to result in a concentration of 2 mg/mL. Since slight precipitation was observed, the solution was centrifuged and a supernatant was used for the measurement. The conditions for measurement were as follows:

Measurement instrument: JASCOJ-500A

Solvent: water

Protein concentration: approximately 2 mg/mL

Wavelength range: 200 to 250 nm

Cell length: 1 mm

Temperature: room temperature (approximately 23° C.)

Number of accumulation: 8

The obtained CD spectrum is shown in FIG. 4. The CD value (vertical axis) is represented by ellipticity (mdeg). Though ordered secondary structures such as α-helix were present to some extent, unordered structures were presumed to be the main structures.

(8) Optical Rotation

The optical rotation was 42, which was measured at 25° C.

(9) Infrared Spectroscopic Analysis

Infrared spectroscopic analysis was conducted by the KBr method. More specifically, 0.5 mg of the M2 fraction and 15 mg of KBr powder were homogeneously mixed, and then molded into a disk shape by pressing for the measurement.

The obtained spectrum is shown in FIG. 5. This spectrum suggests that the M2 fraction contained polysaccharides.

(10) Ultraviolet Spectroscopic Analysis (UV)

The M2 fraction was dissolved in pure water so as to have a concentration of 0.5 mg/10 mL, which was used for the measurement. As an instrument, 2500PC (Shimadzu Corp.) was used.

The obtained UV-visible absorption spectrum is shown in FIG. 6. In the wavelengths from 260 to 270 nm, a weak absorption maximum was observed.

(11) Electron Spin Resonance (ESR)

Using ESP350E (Brucker), ESR of a sample was measured under a nitrogen atmosphere. The main operation conditions are shown in Table 5.

The results are shown in Table 6 and FIGS. 7 and 8. In FIGS. 7 and 8, the strength (arb.units) of the vertical axes means that the unit of strength indicated by the vertical axes is arbitrary. Signals that were considered attributable to carbon radicals were observed around g=2.004. Further, signals around g=4.25 (Fe³⁺) and g=2.03 to 2.05 were considered attributable to transitional metal ions.

TABLE 5

| Conditions | Broad area | Around g = 2 |
|---|---|---|
| Measurement tempera- | room temperature | room temperature |
| ture | | |
| Magnetic field sweep | 0 to 1 T | 339.0 to 359.0 mT |
| area | | |
| Modulation | 100 kZ, 0.5 mT | 100 kZ, 0.2 mT |
| Microwave | 10 mW, 9.79 GHz | 0.2 mW, 9.79 GHz |
| Sweeping period | 167.772 s × 1 time | 83.886 s × 10 times |
| Time constant | 163.84 ms | 163.84 ms |
| Data point number | 4096 points | 2048 points |
| Cavity | TM₁₁₀, cylinder type | TM₁₁₀, cylinder type |

TABLE 6

| Index | M2 fraction | m2 fraction |
|---|---|---|
| g value | 2.0042 | 2.0039 |
| Line width (mT) | 0.68 | 0.67 |
| Spin density | 4.7 × 10¹⁶ | 2.8 × 10¹⁶ |
| (spins/g) | | |

(12) Viscosity

0.5 g of sample (M2 fraction or m2 fraction) was dissolved in 100 mL of purified water, and the solution was centrifuged at 10000 rpm for separation. A supernatant thereof was collected and adjusted so as to have a concentration of 1.67 mg/mL with purified water. Thereafter, the reduced viscosity was measured at 30° C. using an Ostwald viscometer. The reduced viscosities of the M2 fraction and m2 fraction were 108 η and 924 η, respectively.

(13) Molecular Weight

A sample (M2 fraction or m2 fraction) was dissolved in purified water so as to have a concentration of 2 to 3 mg/mL, and gel filtration was performed under the following conditions. Elution times were extrapolated to standard curves with known molecular weights to calculate molecular weights.

Instrument: liquid chromatograph pump LC-7A (Shimadzu Corp.)

Detector: UV spectrophotometric detector SPD-6A (Shimadzu Corp.)

Column: TSK-gel G3000SW (7.5 mm I.D.×30 cm; Tosoh Corp.)

Column temperature: room temperature

Mobile phase: 50 mmol/L phosphate buffer system containing 0.15 mol/L sodium sulfate (pH 7.0)

Flow rate of mobile phase: 0.8 mL/min.

Wavelength for detection: 214 nm

The major component of the M2 fraction had a molecular weight of 2000 kDa and components having 4.0 kDa and 1.2 kDa were also confirmed. The major component of the m2 fraction had a molecular weight of 2000 kDa, and components having 7.0 kDa and 1.0 kDa were also confirmed.

(14) Elementary Analysis

Carbon (C), hydrogen (H), and nitrogen (N) were measured using an organic and trace element analyzer (Yanaco CHN corder TM-5).

Further, with respect to sulfur (S), phosphorus (P), and chlorine (Cl), a sample was combusted and decomposed by a bomb method (O₂ gas), and then SO₄²⁻, PO₄³⁻, and Cl⁻ in the adsorption solution were measured by an ion chromatography (IC) method. The measured values were used to determine the amount of each element. Specifically, 1 mL of acetone was added to 0.1 g of sample and oxygen (3 Mpa) was introduced into the solution. Then, the solution was combusted and cooled with water for 30 minutes. After 0.1 mol/L-NaOH adsorption solution and a washing solution were added to resultant product until the total volume became 100 mL, measurement was conducted using a Dionex DX-300 IC. The results are shown in Table 7.

TABLE 7

Content (mass %)

| Element | M2 fraction | m2 fraction |
|---|---|---|
| Carbon | 41.3 | 40.4 |
| Hydrogen | 6.0 | 6.0 |
| Nitrogen | 5.1 | 8.0 |
| Sulfur | 1.0 | 0.22 |
| Phosphorus | 0.052 | 0.096 |
| Chlorine | 0.16 | 0.13 |

(15) α-glucan Estimated Content

A sample (M2 fraction or m2 fraction) was dissolved in 0.5 mol/L acetic acid buffer (pH 4.3), and amyloglucosidase solution (Sigma Chem. Co.) was added thereto. The resultant solution was shaken at 60° C. for 30 minutes. Next, the solution was adjusted to pH 4.5, and then glucoamylase (Wako Pure Chemical Industries, Ltd.) was added thereto. The resultant solution was shaken at 60° C. for 30 minutes. After the completion of the reaction, the glucose amount in each of the obtained reaction solutions was measured by a glucose measurement instrument. The obtained glucose amount was subtracted from the glucose amount of the blank solution, and the obtained value was defined as the “a-glucan estimated amount.” Additionally, 1.0 mol/L sulfuric acid was added to a sample, and the mixture was hydrolyzed at 100° C. for 18 hours and then neutralized. The glucose amount of each of the obtained reaction solutions was measured by the glucose measurement instrument, and the obtained value was defined as the “total glucan amount.” The α-glucan estimated content was calculated by dividing the “α-glucan estimated amount” by the “total glucan amount,” and multiplying the resultant value by 100.

The α-glucan estimated contents of the M2 fraction and the m2 fraction were 71% and 32%, respectively, based on the total carbohydrates.

(16) Determination of Endotoxin

Using a commercially available kit (ENDOSPECY; Seikagaku Corp.), endotoxin-free apparatuses, and reagents (Seikagaku Corp.), the amount of endotoxin was determined by LAL (Limulus Amoebocyte Lysate) reaction (Ohbayashi T. et al., “Clin. Chem. Acta,” 149, 55-65, 1985).

After the M2 fraction was dissolved in distilled water so as to have a proper concentration, 50 μL of the solution was dispensed into an endotoxin-free 96-well microplate. Into other wells, distilled water or dilution series of endotoxin standard solution was dispensed at the same volume as the above solution. Next, 50 μL of LAL solution (horseshoe crab-derived reagent) was dispensed into each well of the microplate, and the solution mixtures were incubated at 37° C. for 30 minutes. Diazo coupling reagent was then added to the mixtures for color development, and the absorbance was measured at the wavelength of 545 nm (control 630 nm). Based on a curve of the standard solution, the endotoxin amount of the M2 fraction was calculated, and the amount thereof was 2.5 ng/mg.

### Example 7

[Preparation of Anion Exchange Resin Adsorption Fraction of Liquid Extract Mixture of Commercially Available matsutake]

100 g of commercially available matsutake fruit body grown in Nagano, Japan was lyophilized for moisture removal, and then ground to obtain 15 g of powder.

Hereinafter, the extraction and fractionation operations of Example 1 were repeated except that the above fruit body power, instead of mycelia, was used as a starting material, thereby obtaining a non-adsorption fraction m1 and an adsorption fraction m2.

The m2 fraction was also found to have infection resistance to pathogenic bacteria, though its resistance was weaker than that of the M2 fraction.

As described in detail above, the infection preventive or therapeutic agent and food of the present invention can prevent infection with pathogenic bacteria such as Pseudomonas aeruginosa and Listeria monocytogenes. In addition, even when infectious diseases have been contracted, the agent or food of the present invention enables effective treatment of such diseases. Further, the combination intake of antibiotics with the agent or food of the present invention can reduce the necessary dose of the antibiotics.

What is claimed is:

1. A preventive or therapeutic agent for infection(s) with a pathogenic microorganism, containing a member of basidiomycetes belonging to genus Tricholoma or an extract thereof.

2. The infection preventive or therapeutic agent according to claim 1, wherein the member of basidiomycetes belonging to genus Tricholoma is Tricholoma matsutake.

3. The infection preventive or therapeutic agent according to claim 2, wherein Tricholoma matsutake is a mycelium, a broth or a fruit body (including a spore).

4. The infection preventive or therapeutic agent according to claim 2, wherein Tricholoma matsutake is the FERM BP-7304 strain.

5. The infection preventive or therapeutic agent according to claim 2, wherein the Tricholoma matsutake extract contains at least one of: a hot water extract of the mycelia of FERM BP-7304 strain; an alkaline solution extract of the mycelia of FERM BP-7304 strain; an anion exchange resin adsorption fraction of the hot water extract; and an anion exchange resin adsorption fraction of the alkaline solution extract.

6. The infection preventive or therapeutic agent according to claim 2, wherein the Tricholoma matsutake extract is an anion exchange resin adsorption fraction of a mixture obtained by mixing a hot water extract of the mycelia of the FERM BP-7304 strain with an alkaline solution extract of a mycelia residue when the mycelia hot water extract is obtained, wherein the anion exchange resin adsorption fraction has (a) a carbonhydrate content of 60% to 72% in glucose equivalent determined by a phenol-sulfuric acid method and (b) a protein content of 28% to 40% in albumin equivalent determined by a copper-Folin method.

7. The infection preventive or therapeutic agent according to claim 1, which is intended for pathogenic bacteria.

8. The infection preventive or therapeutic agent according to claim 1, which is intended for a gram-negative bacterium.

9. The infection preventive or therapeutic agent according to claim 8, wherein the gram-negative bacterium is any of Pseudomonas aeruginosa, Escherichia coli including O-157, and Helicobacter pylori.

10. The infection preventive or therapeutic agent according to claim 1, which is intended for a gram-positive bacterium.

11. The infection preventive or therapeutic agent according to claim 10, wherein the gram-positive bacterium is Listeria monocytogenes.

12. A preventive or therapeutic agent for infection with a pathogenic microorganism, containing an infection preventive or therapeutic agent of claim 1 and an antibiotic.

13. A method of preventing or treating infection(s) with a pathogenic microorganism which comprises administrating to a human or an animal in an effective amount of the preventive or therapeutic agent of any one of claims 1-11.

14. A method of preventing or treating infection(s) with a pathogenic microorganism which comprises administrating to a human or an animal in an effective amount of the preventive or therapeutic agent of claim 12.

15. A preventive or therapeutic food for infection with a pathogenic microorganism, containing a member of basidiomycetes belonging to genus Tricholoma or an extract thereof.

16. The infection preventive or therapeutic food according to claim 15, wherein the member of basidiomycetes belonging to genus Tricholoma is Tricholoma matsutake.

17. The infection preventive or therapeutic food according to claim 16, wherein a mycelium, a broth or a fruit body (including a spore) of Tricholoma matsutake is used.

18. The infection preventive or therapeutic food according to claim 16, wherein Tricholoma matsutake is the FERM BP-7304 strain.

19. The infection preventive or therapeutic food according to claim 16, wherein the Tricholoma matsutake extract-contains at least one of: a hot water extract of the mycelia of FERM BP-7304 strain; an alkaline solution extract of the mycelia of FERM BP-7304 strain; an anion exchange resin adsorption fraction of the hot water extract; and an anion exchange resin adsorption fraction of the alkaline solution extract.

20. The infection preventive or therapeutic food according to claim 16, wherein the Tricholoma matsutake extract is an anion exchange resin adsorption fraction of a mixture solution obtained by mixing a hot water extract of the mycelia of the FERM BP-7304 strain with an alkaline solution extract of a mycelia residue when the mycelia hot water extract is obtained, wherein the anion exchange resin adsorption fraction has (a) a carbohydrate content of 60% to 72% in glucose equivalent determined by a phenol-sulfuric acid method and (b) a protein content of 28% to 40% in albumin equivalent determined by a copper-Folin method.

21. The infection preventive or therapeutic food according to claim 15, which is intended for pathogenic bacteria.

22. The infection preventive or therapeutic food according to claim 15, which is intended for a gram-negative bacterium.

23. The infection preventive or therapeutic food according to claim 22, wherein the gram-negative bacterium is any of Pseudomonas aeruginosa, Escherichia coli including O-157, and Helicobacter pylori.

24. The infection preventive or therapeutic food according to claim 15, which is intended for a gram-positive bacterium.

25. The infection preventive or therapeutic food according to claim 24, wherein the gram-positive bacterium is Listeria monocytogenes.

26. A preventive or therapeutic food for infection with a pathogenic microorganism, containing an infection preventive or therapeutic food of claim 15 and an antibiotic.

27. A method of preventing or treating infection(s) with a pathogenic microorganism which comprises the intake of by a human or an animal in an effective amount of the preventive or therapeutic food of any one of claims 15-25.

28. A method of preventing or treating infection(s) with a pathogenic microorganism which comprises the intake of by a human or an animal in an effective amount of the preventive or therapeutic food of claim 26.
